(19) United States
(12) Patent Application Publication
(10) Pub. No.: US 2026/0167590 A1
(43) Pub. Date: Jun. 18, 2026

(54) SYSTEMS AND METHODS FOR PLASTIC WASTE RECYCLING

(71) Applicants: Yuxin Wang, Morgantown, WV (US); Jianli Hu, Morgantown, WV (US); Chunlin Luo, Morgantown, WV (US)

(72) Inventors: Yuxin Wang, Morgantown, WV (US); Jianli Hu, Morgantown, WV (US); Chunlin Luo, Morgantown, WV (US)

(21) Appl. No.: 19/424,630

(22) Filed: Dec. 18, 2025

Related U.S. Application Data
(60) Provisional application No. 63/735,517, filed on Dec. 18, 2024.

(51) Int. Cl. C07C 4/06; B01J 8/00; B01J 8/02

(52) U.S. Cl. CPC C07C 4/06; B01J 8/0015; B01J 8/0278; B01J 8/0285; B01J 2208/00442; B01J 2208/00752; B01J 2208/026

(57) ABSTRACT

In one aspect, the disclosure relates to a method comprising: contacting an M1/support catalyst with a plastic feedstock in a reaction zone; purging the reaction zone with an inert gas; and heating the M1/support catalyst by microwave irradiation to a target temperature, thereby producing a gaseous product. The disclosure additionally relates to a system comprising: a microwave chamber; a microwave generator; a sample feeder; a gas delivery device; and a condenser. In one aspect, the system disclosed herein can be used to perform any one of the methods disclosed herein. This abstract is intended as a scanning tool for purposes of searching in the particular art and is not intended to be limiting of the present disclosure.

### CROSS REFERENCE TO RELATED APPLICATIONS

This application claims the benefit of and priority to U.S. Provisional Application No. 63/735,517, filed on Dec. 18, 2024 which is incorporated herein by reference in its entirety.

### BACKGROUND

The global plastic waste crisis has reached a critical point, with plastic waste management (e.g., polyolefin thermoplastic management) contributing to environmental challenges. Polyolefins thermoplastics include polymers such as polyethylene and polypropylene. Propylene, a key molecule in the petrochemical industry, continues to see rising demand. Global production capacity is projected to increase alongside expected growth in market value. Conventional methods for propylene production, such as steam and catalytic cracking of naphtha, are insufficient to meet this growing demand. Efficiently converting polypropylene waste and other plastic waste into useful products, such as propylene, offers a solution to address both the plastic waste crisis and the supply gap.

Traditional chemical recycling or upcycling methods, such as pyrolysis, solvolysis, hydrogenolysis, and hydrocracking, typically require high energy input, operate under H₂ input and high pressure conditions, and mainly produce low-value products, often accompanied by CO₂ emissions. Even with advanced techniques such as electrified spatiotemporal heating and rapid pulse Joule heating, propylene yields from polypropylene depolymerization are relatively low. Therefore, there is a need for efficient and less mostly methods and systems for upcycling plastic waste. These needs and other needs are satisfied by the present disclosure.

### SUMMARY

In accordance with the purpose(s) of the disclosure, as embodied and broadly described herein, the disclosure, in one aspect, relates to methods comprising: contacting an M1/support catalyst with a plastic feedstock in a reaction zone; purging the reaction zone with an inert gas; and heating the M1/support catalyst by microwave irradiation to a target temperature, thereby producing a gaseous product; wherein the support catalyst is selected from zeolite, CeO₂, ZrO₂, and α-Fe₂O₃; wherein M1 is a metal nanoparticle selected from Zr, Ga, Cs, Pt, Pd, Ru, and a combination thereof.

Also disclosed are systems comprising: a microwave chamber, comprising a catalyst, a microwave inlet, a sample inlet, a gas inlet, and an outlet; a microwave generator, configured to generate microwave radiation and deliver the microwave radiation to the microwave chamber via the microwave inlet; a sample feeder, comprising a plastic feedstock and configured to deliver the plastic feedstock from to the microwave chamber via the sample inlet; a gas delivery device, comprising an inert gas and in gaseous communication with the microwave generator via the gas inlet; and a condenser, in gaseous communication with the microwave chamber via the outlet and configured to collect a gaseous product produced in the microwave chamber. In one aspect, the systems disclosed herein can be used to perform any of the methods disclosed herein.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows a representative experimental set-up schematic of microwave-assisted catalytic upcycling of plastic wastes.

FIGS. 1B-1C show representative comparisons of gas product percentages (FIG. 1B) and composition selectivity in gas products (FIG. 1C) between thermal heating methods at temperatures of 500° C., 600° C., and 700° C. and a microwave heating method at a temperature of 400° C.

FIGS. 1D-1E show a representative reaction heating process for microwave and thermal heating (FIG. 1D) and a representative time on stream C₃H₆ flow during the reaction process (FIG. 1E).

FIG. 2A shows representative images depicting post-commodity polypropylene cup wastes from major fast-food brands.

FIGS. 2B-2C show performance of a representative direct microwave catalytic upcycling method for the upcycling of post-consumer polypropylene cup wastes over a Ru (4 wt. %)/α-Fe₂O₃ catalyst.

FIGS. 2D-2E show stability performance of a representative direct microwave catalytic upcycling system.

FIG. 2F shows a representative comparison of the C₃H₆ monomer yield between a representative direct microwave catalytic upcycling system and alternative processes.

FIGS. 3A-3B show representative thermogravimetric analysis (FIG. 3A) and derivative thermogravimetric analysis (FIG. 3B) of different PP feedstocks.

FIGS. 3C-3D show representative thermogravimetric analysis (FIG. 3C) and derivative thermogravimetric analysis (FIG. 3D) of post-reaction residues following direct microwave catalytic upcycling.

FIGS. 4A-4E show representative transmission electron microscopy images of a fresh Ru/α-Fe₂O₃ catalyst (FIGS. 4A-4C) and spent Ru/α-Fe₂O₃ catalyst (FIGS. 4D-4E).

FIG. 5 shows representative Raman spectra of fresh Ru/α-Fe₂O₃, spent Ru/α-Fe₂O₃ in pure chemical PP, and spent Ru/α-Fe₂O₃ in real-world PP.

FIGS. 6A-6C show a comparison of representative X-ray photoelectron spectra of fresh and spent Ru/α-Fe₂O₃ for Fe 2p core level (FIG. 6A), Ru 3d core level (FIG. 6B), and O 1s core level (FIG. 6C).

FIGS. 7A-7B show a representative comparison of different phase products percentage (FIG. 7A) and composition selectivity in gas products (FIG. 7B) between sole Fe₂O₃ and Ru/α-Fe₂O₃ under microwave heating 400° C.

FIG. 7C shows representative reaction pathways using Ru/α-Fe₂O₃ as a catalyst under microwave irradiation.

FIGS. 8A-8B show representative reaction pathways on sole α-Fe₂O₃ catalyst (FIG. 8A) and Ru (4 wt. %)/Fe₂O₃ catalyst (FIG. 8B) under microwave irradiation.

FIG. 9A shows representative X-ray diffraction spectra of a fresh Ru/Fe₂O₃ catalyst, a sole α-Fe₂O₃ catalyst (labeled in the figure as pure α-Fe₂O₃), and the standard JCPDS #02-0919 reference pattern.

FIGS. 9B-9C show representative Raman spectra (FIG. 9B) and X-ray photoelectron spectra (FIG. 9C) of a fresh Ru/Fe₂O₃ catalyst, a sole α-Fe₂O₃ catalyst (labeled in the figures as pure α-Fe₂O₃), and a spent Ru/Fe₂O₃ catalyst.

FIG. 9D shows representative temperature-programmed desorption of ammonia for a fresh Ru/Fe₂O₃ catalyst and a sole α-Fe₂O₃ catalyst (labeled in the figure as pure α-Fe₂O₃).

FIG. 9E shows representative in situ Fourier-transform infrared spectra of pure polypropylene depolymerization using a Ru/Fe₂O₃ catalyst.

FIG. 9F shows representative in situ Raman spectra of pure polypropylene depolymerization using a Ru/Fe₂O₃ catalyst

FIGS. 10A-10B shows representative calculated energy barriers and configurations for C—H (FIG. 10A) and C—C dissociation (FIG. 10B) on both sole α-Fe₂O₃ and Ru/Fe₂O₃.

FIGS. 10C-10D show representative projected density of states of Fe—O (FIG. 10C) and Ru—O (FIG. 10D).

FIGS. 10E-10H show representative charge density differences of simplified saturated long chain molecule (CH₃CH₃CHCH₂CH₂CH₃, FIG. 10E and FIG. 10F) and unsaturated long chain molecules (CH₃CH₃CCHCH₂CH₃, FIG. 10G and FIG. 10H) adsorbed on sole α-Fe₂O₃ and Ru/Fe₂O₃. The isosurface level is set 0.002 electron/bohr3. Yellow and blue colors represent the electron accumulation and consumption regions, respectively.

FIG. 10I shows representative proposed reaction pathways on the surface of sole α-Fe₂O₃.

FIG. 10J shows representative proposed reaction pathways on the surface of Ru/α-Fe₂O₃

FIG. 11 shows representative microwave heating process curves at 400° C.

FIGS. 12A-12F show representative thermal images of a Ru/Fe₂O₃ catalyst and polypropylene particle mixture at different temperatures during a microwave heating process.

FIG. 13 shows a representative schematic of a continuous feed microwave reactor system for the upcycling of plastic waste.

FIG. 14 shows a representative photograph of a continuous feed microwave reactor system for the upcycling of plastic waste.

FIGS. 15A-15G show representative close-up photographs of the system depicted in FIG. 14, including a microwave control program (FIG. 15A), a microwave generator (FIG. 15B), a sample feeder and component for continuous feeding of plastic waste sample (FIG. 15C and FIG. 15D), a microwave chamber (FIG. 15E), a cold trap (FIG. 15F), and a close-up of the sample-feeder's connection to the microwave chamber (FIG. 15G).

FIGS. 16A-16F show SEM-EDX mappings of a representative spent catalyst.

FIG. 17 shows a representative reaction scheme at the interface between a catalyst and a PP under a DMCU system.

FIGS. 18A-18B show a comparison of gas products percentage (FIG. 18A) and composition selectivity in gas products (FIG. 18B) using different Ru loading catalysts at microwave heating 400° C.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Reference to “a” chemical compound refers to one or more molecules of the chemical compound rather than being limited to a single molecule of the chemical compound. Furthermore, the one or more molecules may or may not be identical, so long as they fall under the category of the chemical compound. Thus, for example, “a” chemical compound is interpreted to include one or more molecules of the chemical, where the molecules may or may not be identical (e.g., different isotopic ratios, enantiomers, and the like).

As used in the specification and the appended claims, the singular forms “a,” “an” and “the” include plural referents unless the context clearly dictates otherwise. Thus, for example, reference to “a polyolefin,” “an inert gas,” or “a catalyst,” includes, but is not limited to, two or more such polyolefins, inert gases, or catalysts, and the like.

Reference to “a/an” chemical compound each refers to one or more molecules of the chemical compound rather than being limited to a single molecule of the chemical compound. Furthermore, the one or more molecules may or may not be identical, so long as they fall under the category of the chemical compound.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “contacting” as used herein refers to bringing a disclosed analyte, compound, chemical, or material in proximity to another disclosed analyte, compound, chemical, or material as indicated by the context. In some instances, contacting can comprise both physical and chemical interactions between the indicated components. It is to be understood that chemical interactions can comprise a combination of covalent and non-covalent interactions, including one or more of ionic, dipolar, van der Waals interactions, and the like. For example, a plastic feedstock contacting a catalyst is understood to mean that the plastic feedstock is at least in physical contact with the catalyst.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkene” or “olefin” as used herein refers to a hydrocarbon compound of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. An alkene compound can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

As used herein, the term “light olefin” refers to an olefin of 2 to 4 carbon atoms (i.e., a C2-C₄ alkene).

As used herein, the term “polyolefin” or “polyolefin plastic” refers to thermoplastic polymers with the general formula —(CH₂CHA¹)ₙ-, where n is an integer and A¹ is a hydrogen or an alkyl group as defined herein. Examples of thermoplastic polymers include polyethylene (e.g., low-density polyethylene, linear low-density polyethylene, and high-density polyethylene) and polypropylene.

Unless otherwise specified, temperatures referred to herein are based on atmospheric pressure (i.e. one atmosphere).

B. ABBREVIATIONS

| | |
|---|---|
| BTX | aromatics |
| DFT | density functional theory |
| DMCU | direct microwave catalytic upcycling |
| EDX | energy-dispersive X-ray spectroscopy |
| FTIR | Fourier-transform infrared |
| MW | microwave |
| PP | polypropylene |
| SEM | scanning electron microscopy |
| TEM | transmission electron microscope |
| TGA | thermogravimetric analysis |
| TH | thermal heating |
| TPD | temperature programmed desorption |

### C. INTRODUCTION

Plastic wastes such as polypropylene contribute significantly to environmental challenges. PP, which accounts for 14% of total polymer production, is projected to rise from 79.01 in 2022 to 104.99 million metric tons by 2030 (Ref. 62). A prime example is the widespread use of single-use PP beverage cups by major food service companies, yet the recovery rate of such waste remains alarmingly low at only 1%, exacerbating the issue of plastic pollution (Refs. 1-3).

Demand for propylene is on the rise, with the global production capacity of propylene projected to increase from 150.30 million metric tons in 2022 to 219.68 million metric tons by 2030, with market value expected to grow from USD 107.96 billion in 2023 to USD 150.54 billion by 2032 (Refs. 4 and 5). Conventional methods for the production of propylene, such as steam and catalytic cracking of naphtha, are insufficient to meet this demand (Ref. 6). Alternative means of producing propylene, such as by converting PP waste into propylene, offer a means of addressing both the supply gap and the plastic waste crisis (Ref. 7).

Landfilling, incineration, and mechanical plastic waste recycling methods lead to environmental pollution, inefficient energy recovery as heat, and lower-value products compared to the original plastics (Refs. 2 and 8). Traditional chemical upcycling methods, such as pyrolysis, solvolysis, hydrogenolysis, and hydrocracking, typically require high energy input, operate under H₂ input and high pressure conditions, and mainly produce low-value products, often accompanied by CO₂ emissions (Ref. 9). Conventional catalytic pyrolysis faces challenges in precisely controlling key processes such as melting, vaporization, decomposition, and competing side reactions during depolymerization. This often results in a broad product distribution, rather than selective production of original monomers like propylene from polypropylene (Refs. 9-11). For instance, catalytic pyrolysis of PP with optimized catalysts typically achieves propylene yields of less than 25% (Ref. 12). Even with advanced techniques such as electrified spatiotemporal heating and rapid pulse Joule heating, propylene yields from PP depolymerization only reach around 36% and 35%, respectively (Refs. 13 and 14). Thus, achieving high propylene selectivity in the direct catalytic upcycling of PP plastic waste under mild conditions remains a significant challenge to date (Ref. 15).

In one aspect, disclosed herein is a method for the recycling or upcycling of plastic waste (e.g., polyolefin waste) using microwave heating. A DMCU process offers a sustainable solution for plastic waste recycling. Unlike conventional heating, microwave heating provides rapid, volumetric energy transfer, improving reaction rates, selectivity, and energy efficiency, especially in endothermic catalytic reactions (Ref. 16 and 17). In a further aspect, this method leverages the interaction between microwave energy and microwave-sensitive catalysts, addressing the limitations of traditional catalytic pyrolysis, which often produces complex mixtures with low selectivity for valuable chemicals (Ref. 18-20). In one aspect, the methods disclosed herein can produce an increased yield of a target product (e.g., propylene) along with fewer by-products compared to conventional upcycling methods. The methods disclosed herein can also be performed at milder conditions (e.g., ambient pressure and a temperature of less than about 500° C.) compared to conventional methods. Integrating a microwave-responsive catalyst design with microwave catalysis for the direct conversion of polyolefin waste into valuable products offers an efficient and sustainable approach to plastic waste recycling or upcycling. Also disclosed herein is a system for recycling or upcycling of plastic waste, which, in one aspect, can allow for a continuous recycling or upcycling of plastic waste.

### D. METHOD FOR PLASTIC WASTE RECYCLING

Disclosed herein is a method for plastic waste recycling or upcycling comprising: contacting an M1/support catalyst with a plastic feedstock in a reaction zone; purging the reaction zone with an inert gas; and heating the M1/support catalyst by microwave irradiation to a target temperature, thereby producing a gaseous product. The reaction zone can be the inside of a reactor, such as a microwave reactor or microwave chamber. The inert gas can be selected from N₂, Ar, CO₂, H₂, He, and a combination thereof. The support catalyst can be selected from zeolite, CeO₂, ZrO₂, and α-Fe₂O₃. A zeolite is a crystalline aluminosilicate material that can be naturally or synthetically formed. Examples of zeolite supports include Y-type zeolite, a pentasil aluminosilicate zeolite (e.g., Zeolite Socony Mobil-5 or ZSM-5), a mordenite (MOR) zeolite, and a beta zeolite. In one aspect, M1 is a metal nanoparticle that can be selected from Zr, Ga, Cs, Pt, Pd, Ru, and a combination thereof.

In one aspect, contacting the M1/support catalyst with the plastic feedstock can comprise contacting a fixed-bed catalyst with the plastic feedstock, such as adding the microwave feedstock to a reactor (e.g., microwave reactor or microwave chamber) comprising a fixed catalyst bed (i.e., a fixed-bed reactor). In another aspect, contacting the M1/support catalyst with the plastic feedstock can comprise mixing the plastic feedstock with the M1/support catalyst. The plastic feedstock and the M1/support catalyst can be mixed in a mass ratio of about 0.25:1 to about 3:1, about 0.25:1 to about 2:1, about 0.25:1 to about 1:1, about 1:3 to about 3:1, about 1:3 to about 2:1, about 1:3 to about 1:1, about 0.5:1 to about 3:1, about 0.5:1 to about 2:1, about 0.5:1 to about 1:1, about 0.5:1 to about 1:0.5, about 0.55:1 to about 1:0.55, about 0.6:1 to about 1:0.6, about 0.65:1 to about 1:0.65, about 0.75:1 to about 1:0.75, about 0.8:1 to about 1:0.8, about 0.85:1 to about 1:0.85, about 0.9:1 to about 1:0.9, about 0.95:1 to about 1:0.95, or about 1:1.

In one aspect, the plastic feedstock can be obtained by processing plastic wastes obtained from packaging materials, storage containers, food packaging, food containers, electronics, machinery, and the like. In one aspect, the plastic feedstock can include a polyolefin plastic, such as polypropylene, polyethylene, or a derivative thereof. In another aspect, the plastic feedstock can include a blend of various polymers, including polylactic acid, thermoplastic starch, polyolefins such as polypropylene and polyethylene, and the like. The plastic feedstock can be prepared by crushing and/or grinding plastic waste into smaller pieces, using a device such as a plastic pulverizer. Optionally, the resulting ground plastic waste can then be sieved to a desired particle size. In one aspect, the plastic feedstock can comprise particles of plastic, where the particles of plastic have a particle size of less than about 10 mm, less than about 8 mm, less than about 6 mm, less than about 5 mm, or less than about 2 mm. In another aspect, the particles of plastic can have a particle size of about 0.25 mm to about 10 mm, about 0.25 mm to about 8 mm, about 0.25 mm to about 6 mm, about 0.25 mm to about 5 mm, about 0.25 mm to about 4 mm, about 0.25 mm to about 2 mm, about 1 mm to about 8 mm, about 1 mm to about 6 mm, or about 1 mm to about 4 mm. In another aspect, the plastic feedstock can be melted prior to its incorporation into the methods disclosed herein and be used in melt or liquid form.

The M1/support catalyst can be prepared by loading the support catalyst with the M1 metal nanoparticle. The catalyst can be loaded with the metal nanoparticle at a weight percentage of about 1% to about 6%, about 1% to about 5%, about 1% to about 4%, about 2% to about 6%, about 2% to about 5%, or about 3% to about 5%. The metal nanoparticles can be various shapes, such as spheres, cubes, rods, wires, polygonal plates, amorphous, polyhedrons, and the like, or combinations thereof. Any size of nanoparticles can be used to produce the catalyst. For example, the nanoparticles can range in size from about 100 nm to about 2000 nm, about 100 nm to about 1000 nm, about 100 nm to about 500 nm, about 500 nm to about 2000 nm, about 1000 nm to about 2000 nm, or about 1500 nm to about 2000 nm.

In a further aspect, the M1/support catalyst can be heated by microwave irradiation to a target temperature of less than about 600° C., less than about 550° C., less than about 500° C., less than about 450° C., or less than about 400° C. In another aspect, the target temperature can be about 300° C. to about 600° C., about 300° C. to about 550° C., about 300° C. to about 500° C., about 300° C. to about 450° C., about 300° C. to about 400° C., about 350° C. to about 500° C., about 400° C. to about 500° C., or about 350° C. to about 450° C. The M1/support catalyst can be heated by microwave irradiation at a rate of about 10° C./min to about 30° C./min, about 10° C./min to about 25° C./min, about 10° C./min to about 20° C./min, about 15° C./min to about 30° C./min, about 15° C./min to about 25° C./min, or about 20° C./min to about 30° C./min until reaching the target temperature. After reaching the target temperature, the M1/support catalyst can be held at the target temperature for about 0.5 minutes to about 1 hour, about 0.5 minutes to about 45 minutes, about 0.5 minutes to about 30 minutes, about 1 minutes to about 1 hour, about 1 minutes to about 45 minutes, about 1 minutes to about 30 minutes, about 5 minutes to about 1 hour, about 5 minutes to about 45 minutes, about 5 minutes to about 30 minutes, about 15 minutes to about 1 hour, or about 15 minutes to about 45 minutes.

The gaseous product produced using any one of the methods disclosed herein can comprise a light olefin gas, such propylene gas. The light olefin gas can comprise at least 40 wt %, at least 45 wt %, at least 50 wt %, at least 55 wt %, or at least 60 wt % of the gaseous product. In another aspect, the light olefin gas can comprise from about 30 wt % to about 100 wt %, about 30 wt % to about 90 wt %, about 30 wt % to about 80 wt %, about 30 wt % to about 70 wt %, about 40 wt % to about 100 wt %, about 40 wt % to about 90 wt %, about 40 wt % to about 80 wt %, about 40 wt % to about 70 wt %, about 40 wt % to about 60 wt %, about 50 wt % to about 100 wt %, about 50 wt % to about 90 wt %, about 50 wt % to about 80 wt %, about 50 wt % to about 70 wt %, or about 50 wt % to about 60 wt % of the gaseous product. In one aspect, the methods disclosed herein can produce an increased yield of a desired target product (e.g., light olefin gas such as propylene). Product yield, as used herein, is calculated as the ratio of the mass of product produced (e.g., light olefin gas produced) to the mass of the plastic feedstock. The light olefin gas can have a product yield of at least 30%, at least 35%, at least 40%, at least 45%, or at least 50%. In another aspect, the product yield for light olefin gas can range from about 40% to about 100%, about 40% to about 90%, about 40% to about 80%, about 40% to about 70%, about 40% to about 60%, about 50% to about 100%, about 50% to about 90%, about 50% to about 80%, or about 50% to about 70%.

### E. SYSTEMS FOR PLASTIC WASTE RECYCLING

Also disclosed herein are systems that can be used in the recycling or upcycling of plastic waste. The systems can be used to perform any one of the methods disclosed herein. In one aspect, the system, comprises: a microwave chamber, comprising a catalyst, a microwave inlet, a sample inlet, a gas inlet, and an outlet; a microwave generator, configured to generate microwave radiation and deliver the microwave radiation to the microwave chamber via the microwave inlet; a sample feeder, comprising a plastic feedstock and configured to deliver the plastic feedstock from to the microwave chamber via the sample inlet; a gas delivery device, comprising an inert gas and in gaseous communication with the microwave generator via the gas inlet; and a condenser, in gaseous communication with the microwave chamber via the outlet and configured to collect a gaseous product produced in the microwave chamber. The inert gas can be selected from N₂, Ar, CO₂, H₂, He, and a combination thereof.

In one aspect, the catalyst can be an M1/support catalyst. The support catalyst can be selected from zeolite, CeO₂, ZrO₂, and α-Fe₂O₃. Examples of zeolite supports include Y-type zeolite, a pentasil aluminosilicate zeolite (e.g., Zeolite Socony Mobil-5 or ZSM-5), a mordenite (MOR) zeolite, and a beta zeolite. In one aspect, M1 is a metal nanoparticle that can be selected from Zr, Ga, Cs, Pt, Pd, Ru, and a combination thereof. The M1/support catalyst can be prepared by loading the support catalyst with the M1 metal nanoparticle. The catalyst can be loaded with the metal nanoparticle at a weight percentage of about 1% to about 6%, about 1% to about 5%, about 1% to about 4%, about 2% to about 6%, about 2% to about 5%, or about 3% to about 5%. The metal nanoparticles can be various shapes, such as spheres, cubes, rods, wires, polygonal plates, amorphous, polyhedrons, and the like, or combinations thereof. Any size of nanoparticles can be used to produce the catalyst. For example, the nanoparticles can range in size from about 100 nm to about 2000 nm, about 100 nm to about 1000 nm, about 100 nm to about 500 nm, about 500 nm to about 2000 nm, about 1000 nm to about 2000 nm, or about 1500 nm to about 2000 nm.

The system can further comprise a pyrometer, such as an infrared pyrometer. In one aspect, plastic feedstock delivered to the microwave chamber can form a reaction mixture with the catalyst. The pyrometer can be configured to monitor the temperature of the catalyst, plastic feedstock, and/or the reaction mixture in the microwave chamber.

### F. REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
Ref 1. (Statista Research Department, 2023).
Ref 2. A. Rahimi, J. M. García, Chemical recycling of waste plastics for new materials production. Nature Reviews Chemistry 1, 0046 (2017).
Ref 3. S. L. Nordahl, N. R. Baral, B. A. Helms, C. D. Scown, Complementary roles for mechanical and solvent-based recycling in low-carbon, circular polypropylene. Proceedings of the National Academy of Sciences 120, e2306902120 (2023).
Ref 4. Global Propylene Capacity and Capital Expenditure Outlook Report 2023-2030: China is Expected to Lead Propylene Capacity Growth, Followed by India and Iran, Research and Markets, 2023.
Ref 5. Propylene Market Size To Hit USD 150.54 Billion by 2032, Precedence research, 2023.
Ref 6. R. J. Conk et al., Catalytic deconstruction of waste polyethylene with ethylene to form propylene. Science 377, 1561-1566 (2022).
Ref 7. G. W. Coates, Y. D. Getzler, Chemical recycling to monomer for an ideal, circular polymer economy. Nature Reviews Materials 5, 501-516 (2020).
Ref 8. A. J. Martin, C. Mondelli, S. D. Jaydev, J. Perez-Ramirez, Catalytic processing of plastic waste on the rise. Chem 7, 1487-1533 (2021).
Ref 9. K. Zheng et al., Progress and perspective for conversion of plastic wastes into valuable chemicals. Chemical Society Reviews, (2023).
Ref 10. Direct catalytic plastics waste upcycling. Nature Nanotechnology 18, 687 (2023).
Ref 11. S. D. A. Sharuddin, F. Abnisa, W. M. A. W. Daud, M. K. Aroua, A review on pyrolysis of plastic wastes. Energy conversion and management 115, 308-326 (2016).
Ref 12. M. S. Abbas-Abadi et al., Challenges and opportunities of light olefin production via thermal and catalytic pyrolysis of end-of-life polyolefins: Towards full recyclability. Progress in Energy and Combustion Science 96, 101046 (2023).
Ref 13. Q. Dong et al., Depolymerization of plastics by means of electrified spatiotemporal heating. Nature 616, 488-494 (2023).
Ref 14. E. Selvam, K. Yu, J. Ngu, S. Najmi, D. G. Vlachos, Recycling polyolefin plastic waste at short contact times via rapid joule heating. Nature Communications 15, 5662 (2024).
Ref 15. M. Chu, Y. Liu, X. Lou, Q. Zhang, J. Chen, Rational design of chemical catalysis for plastic recycling. ACS Catalysis 12, 4659-4679 (2022).
Ref 16. Y. Wang et al., Microwave-driven upcycling of single-use plastics using zeolite catalyst. Chemical Engineering Journal 465, 142918 (2023).
Ref 17. V. Tuli, C. Luo, B. Robinson, J. Hu, Y. Wang, Microwave-assisted catalytic technology for sustainable production of valuable chemicals from plastic waste with enhanced catalyst reusability. Chemical Engineering Journal 489, 151551 (2024).
Ref 18. X. Jie et al., Microwave-initiated catalytic deconstruction of plastic waste into hydrogen and high-value carbons. Nature Catalysis 3, 902-912 (2020).
Ref 19. J. Serra et al., Hydrogen production via microwave-induced water splitting at low temperature. Nature Energy 5, 910-919 (2020).
Ref 20. A. Ramirez et al., Escaping undesired gas-phase chemistry: Microwave-driven selectivity enhancement in heterogeneous catalytic reactors. Science advances 5, eaau9000 (2019).
Ref 21. H. Shi et al., Gases production from microwave-assisted pyrolysis of polypropylene plastic. Journal of Environmental Chemical Engineering 11, 110851 (2023).
Ref 22. V. Tuli, C. Luo, B. Robinson, J. Hu, Y. Wang, Microwave-assisted catalytic technology for sustainable production of valuable chemicals from plastic waste with enhanced catalyst reusability. Chemical Engineering Journal, 151551 (2024).
Ref 23. P. A. Kots et al., Polypropylene plastic waste conversion to lubricants over Ru/TiO2 catalysts. Acs Catalysis 11, 8104-8115 (2021).
Ref 24. Y.-H. Lin, P. Sharratt, A. Garforth, J. Dwyer, Catalytic conversion of polyolefins to chemicals and fuels over various cracking catalysts. Energy & Fuels 12, 767-774 (1998).
Ref 25. D. S. Scotp, P. Majerski, J. Piskorz, D. Radlein, M. Barnickel, Production of liquid fuels from waste plastics. The Canadian journal of chemical engineering 77, 1021-1027 (1999).
Ref 26. P. Lovás, P. Hudec, B. Jambor, E. Hájeková, M. Horňáček, Catalytic cracking of heavy fractions from the pyrolysis of waste HDPE and PP. Fuel 203, 244-252 (2017).
Ref 27. J. Encinar, J. González, Pyrolysis of synthetic polymers and plastic wastes. Kinetic study. Fuel processing technology 89, 678-686 (2008).
Ref 28. J. M. Arandes, J. Ereña, M. J. Azkoiti, D. López-Valerio, J. Bilbao, Valorization by thermal cracking over silica of polyolefins dissolved in LCO. Fuel processing technology 85, 125-140 (2004).
Ref 29. S.-H. Jung, M.-H. Cho, B.-S. Kang, J.-S. Kim, Pyrolysis of a fraction of waste polypropylene and polyethylene for the recovery of BTX aromatics using a fluidized bed reactor. Fuel processing technology 91, 277-284 (2010).
Ref 30. N. Zhou et al., Catalytic pyrolysis of plastic wastes in a continuous microwave assisted pyrolysis system for fuel production. Chemical Engineering Journal 418, 129412 (2021).
Ref 31. M. S. Abbas-Abadi, M. N. Haghighi, H. Yeganeh, A. G. McDonald, Evaluation of pyrolysis process parameters on polypropylene degradation products. Journal of analytical and applied pyrolysis 109, 272-277 (2014).
Ref 32. G. Lopez, M. Artetxe, M. Amutio, J. Bilbao, M. Olazar, Thermochemical routes for the valorization of waste polyolefinic plastics to produce fuels and chemicals. A review. Renewable and Sustainable Energy Reviews 73, 346-368 (2017).
Ref 33. D. Hassanian-Moghaddam, N. Asghari, M. Ahmadi, Circular polyolefins: Advances toward a sustainable future. Macromolecules 56, 5679-5697 (2023).
Ref 34. F. Kishimoto et al., Direct microwave energy input on a single cation for outstanding selective catalysis. Science Advances 9, eadi1744 (2023).
Ref 35. Y. V. Kissin, Chemical mechanisms of catalytic cracking over solid acidic catalysts: alkanes and alkenes. Catalysis Reviews 43, 85-146 (2001).
Ref 36. H. Ran, S. Zhang, W. Ni, Y. Jing, Precise activation of C—C bonds for recycling and upcycling of plastics. Chemical Science 15, 795-831 (2024).
Ref 37. J. Buchanan, J. Santiesteban, W. Haag, Mechanistic considerations in acid-catalyzed cracking of olefins. Journal of Catalysis 158, 279-287 (1996).
Ref 38. M. Hosseinpour et al., The synergistic effect between supercritical water and redox properties of iron oxide nanoparticles during in-situ catalytic upgrading of heavy oil with formic acid. Isotopic study. Applied Catalysis B: Environmental 230, 91-101 (2018).
Ref 39. J. Li et al., In-situ hydrothermal upgrading and mechanism of heavy oil with nano-Fe2O3 in the porous media. Journal of Analytical and Applied Pyrolysis 183, 106757 (2024).
Ref 40. A. V. Vorontsov, Advancing Fenton and photo-Fenton water treatment through the catalyst design. Journal of hazardous materials 372, 103-112 (2019).
Ref 41. C. W. Yeung, J. Y. Teo, X. J. Loh, J. Y. Lim, Polyolefins and polystyrene as chemical resources for a sustainable future: challenges, advances, and prospects. ACS Materials Letters 3, 1660-1676 (2021).
Ref 42. A. Corma, A. Orchillés, Current views on the mechanism of catalytic cracking. Microporous and mesoporous materials 35, 21-30 (2000).
Ref 43. V. Haridas, A. Sukhananazerin, B. Pullithadathil, B. N. Narayanan, Ultrahigh specific capacitance of α-Fe2O3 nanorods-incorporated defect-free graphene nanolayers. Energy 221, 119743 (2021).
Ref 44. Z. Dong et al., Understanding the structure-activity relationships in catalytic conversion of polyolefin plastics by zeolite-based catalysts: a critical review. ACS catalysis 12, 14882-14901 (2022).
Ref 45. D. D. Hibbitts, D. W. Flaherty, E. Iglesia, Role of branching on the rate and mechanism of C—C cleavage in alkanes on metal surfaces. ACS Catalysis 6, 469-482 (2016).
Ref 46. N. M. Almeida, I. R. Ariyarathna, E. Miliordos, O—H and C—H bond activations of water and methane by RuO2+ and (NH3) RuO2+: ground and excited states. The Journal of Physical Chemistry A 123, 9336-9344 (2019).
Ref 47. G. Kresse, J. Furthmüller, Efficient iterative schemes for ab initio total-energy calculations using a plane-wave basis set. Physical Review B 54, 11169-11186 (1996).
Ref 48. P. E. Blöchl, Projector augmented-wave method. Physical Review B 50, 17953-17979 (1994).
Ref 49. B. Hammer, L. B. Hansen, J. K. Nørskov, Improved adsorption energetics within density-functional theory using revised Perdew-Burke-Ernzerhof functionals. Physical Review B—Condensed Matter and Materials Physics 59, 7413-7421 (1999).
Ref 50. S. Grimme, J. Antony, S. Ehrlich, H. Krieg, A consistent and accurate ab initio parametrization of density functional dispersion correction (DFT-D) for the 94 elements H—Pu. The Journal of chemical physics 132, (2010).
Ref 51. G. Henkelman, B. P. Uberuaga, H. Jónsson, A climbing image nudged elastic band method for finding saddle points and minimum energy paths. The Journal of chemical physics 113, 9901-9904 (2000).
Ref 52. X. Chen et al., Unveiling the Enhancement of Electrocatalytic Oxygen Evolution Activity in Ru—Fe₂O₃/CoS Heterojunction Catalysts. Small, 2403427 (2024).
Ref 53. N. Basavegowda, K. Mishra, Y. R. Lee, Synthesis, characterization, and catalytic applications of hematite (α-Fe2O3) nanoparticles as reusable nanocatalyst. Advances in Natural Sciences: Nanoscience and Nanotechnology 8, 025017 (2017).
Ref 54. D. Serrano, J. Aguado, J. Escola, Developing advanced catalysts for the conversion of polyolefinic waste plastics into fuels and chemicals. Acs Catalysis 2, 1924-1941 (2012).
Ref 55. Q. Zhou et al., Mechanistic Understanding of Efficient Polyethylene Hydrocracking over Two-Dimensional Platinum-Anchored Tungsten Trioxide. Angewandte Chemie International Edition 62, e202305644 (2023).
Ref 56. P. Zhang et al., Sustainable microwave-driven CO2 gasification of plastic waste for high-yield H2 and CO production. Applied Catalysis B: Environment and Energy 345, 123718 (2024).
Ref 57. X. Guo et al., In-line monitoring the degradation of polypropylene under multiple extrusions based on Raman spectroscopy. Polymers 11, 1698 (2019).
Ref 58. V. Mazzieri, F. Coloma-Pascual, A. Arcoya, P. L'Argentière, N. Figoli, XPS, FTIR and TPR characterization of Ru/Al2O3 catalysts. Applied Surface Science 210, 222-230 (2003).
Ref 59. Y. V. Larichev, B. L. Moroz, V. I. Bukhtiyarov, Electronic state of ruthenium deposited onto oxide supports: An XPS study taking into account the final state effects. Applied surface science 258, 1541-1550 (2011).
Ref 60. Y.-H. Lin, P. Sharratt, A. Garforth, J. Dwyer, Catalytic conversion of polyolefins to chemicals and fuels over various cracking catalysts. Energy & Fuels 12, 767-774 (1998).
Ref 61. Zhao, X. Shen, H. Li, K. Liu, H. Wu, X. Li, X. Gao, Watching microwave-induced microscopic hot spots via the thermosensitive fluorescence of europium/terbium mixed-metal organic complexes, Angewandte Chemie International Edition, 61 (2022) e202114340.
Ref 62. Market volume of polypropylene worldwide from 2015 to 2022, with a forecast for 2023 to 2030 (in million metric tons) AgileIntel Research (ChemIntel360), 2023.

### G. ASPECTS

The following listing of exemplary aspects supports and is supported by the disclosure provided herein.

Aspect 1. A method, comprising: contacting an M1/support catalyst with a plastic feedstock in a reaction zone; purging the reaction zone with an inert gas; and heating the M1/support catalyst by microwave irradiation to a target temperature, thereby producing a gaseous product; wherein the support catalyst is selected from zeolite, CeO₂, ZrO₂, and α-Fe₂O₃; wherein M1 is a metal nanoparticle selected from Zr, Ga, Cs, Pt, Pd, Ru, and a combination thereof.

Aspect 2. The method of aspect 1, wherein the M1/support catalyst is a fixed-bed catalyst.

Aspect 3. The method of aspect 1, wherein contacting the M1/support catalyst with the plastic feedstock comprises mixing the plastic feedstock with the M1/support catalyst.

Aspect 4. The method of aspect 2, wherein the plastic feedstock and the M1/support catalyst are mixed in a mass ratio of about 0.25:1 to about 3:1.

Aspect 5. The method of aspect 2, wherein the plastic feedstock and the M1/support catalyst are mixed in a mass ratio of about 1:3 to about 1:1.

Aspect 6. The method of aspect 2, wherein the plastic feedstock and the M1/support catalyst are mixed in a mass ratio of about 0.5:1 to about 1:1.

Aspect 7. The method of any one of aspects 1-5, wherein the plastic feedstock comprises a solid plastic.

Aspect 8. The method of aspect 7, wherein the plastic feedstock comprises particles of plastic.

Aspect 9. The method of aspect 8, wherein the particles of plastic have a particle size of less than about 10 mm.

Aspect 10. The method of aspect 8, wherein the particles of plastic have a particle size of less than about 5 mm.

Aspect 11. The method of aspect 8, wherein the particles of plastic have a particle size of less than about 2 mm.

Aspect 12. The method of aspect 8, wherein the particles of plastic have a particle size of about 0.25 mm to about 10 mm.

Aspect 13. The method of aspect 8, wherein the particles of plastic have a particle size of about 0.25 mm to about 5 mm.

Aspect 14. The method of any one of aspects 1-5, wherein the plastic feedstock comprises a melted plastic.

Aspect 15. The method of any one of aspects 1-14, wherein the inert gas is selected from N₂, Ar, CO₂, H₂, He, and a combination thereof.

Aspect 16. The method of any one of aspects 1-15, wherein the target temperature is less than about 600° C.

Aspect 17. The method of any one of aspects 1-15, wherein the target temperature is less than about 450° C.

Aspect 18. The method of any one of aspects 1-15, wherein the target temperature is less than about 400° C.

Aspect 19. The method of any one of aspects 1-15, wherein the target temperature is about 300° C. to about 500° C.

Aspect 20. The method of any one of aspects 1-15, wherein the target temperature is about 300° C. to about 450° C.

Aspect 21. The method of any one of aspects 1-15, wherein the target temperature is about 300° C. to about 400° C.

Aspect 22. The method of any one of aspects 1-21, wherein the M1/support catalyst is heated at a rate of about 10° C./min to about 30° C./min until reaching the target temperature.

Aspect 23. The method of any one of aspects 1-21, wherein the M1/support catalyst is heated at a rate of about 15° C./min to about 25° C./min until reaching the target temperature.

Aspect 24. The method of any one of aspects 1-23, wherein the M1/support catalyst is held at the target temperature for about 0.5 minutes to about 1 hour.

Aspect 25. The method of any one of aspects 1-23, wherein the M1/support catalyst is held at the target temperature for about 1 minute to about 45 minutes.

Aspect 26. The method of any one of aspects 1-25, wherein the plastic feedstock comprises a polyolefin plastic.

Aspect 27. The method of aspect 26, wherein the polyolefin plastic comprises a polypropylene plastic.

Aspect 28. The method of aspect 26 or aspect 27, wherein the gaseous product comprises a light olefin gas.

Aspect 29. The method of aspect 28, wherein the light olefin gas comprises at least 40 wt % of the gaseous product.

Aspect 30. The method of aspect 28, wherein the light olefin gas comprises at least 50 wt % of the gaseous product.

Aspect 31. The method of aspect 28, wherein the light olefin gas comprises from about 40 wt % to about 100 wt % of the gaseous product.

Aspect 32. The method of aspect 28, wherein the light olefin gas comprises from about 40 wt % to about 90 wt % of the gaseous product

Aspect 33. The method of aspect 28, wherein the light olefin gas comprises from about 50 wt % to about 100 wt % of the gaseous product.

Aspect 34. The method of aspect 28, wherein the light olefin gas comprises from about 50 wt % to about 90 wt % of the gaseous product.

Aspect 35. The method of any one of aspects 28-34, wherein the light olefin gas has a product yield of at least 40%, wherein the product yield is calculated as the ratio of the mass of light olefin gas produced to the mass of the plastic feedstock.

Aspect 36. The method of any one of aspects 28-34, wherein the light olefin gas has a product yield of at least 45%, wherein the product yield is calculated as the ratio of the mass of light olefin gas produced to the mass of the plastic feedstock.

Aspect 37. The method of any one of aspects 28-34, wherein the light olefin gas has a product yield of at least 50%, wherein the product yield is calculated as the ratio of the mass of light olefin gas produced to the mass of the plastic feedstock.

Aspect 38. The method of any one of aspects 28-34, wherein the light olefin gas has a product yield of about 40% to about 100%, wherein the product yield is calculated as the ratio of the mass of light olefin gas produced to the mass of the plastic feedstock.

Aspect 39. The method of any one of aspects 28-34, wherein the light olefin gas has a product yield of about 40% to about 90%, wherein the product yield is calculated as the ratio of the mass of light olefin gas produced to the mass of the plastic feedstock.

Aspect 40. The method of any one of aspects 28-34, wherein the light olefin gas has a product yield of about 50% to about 100%, wherein the product yield is calculated as the ratio of the mass of light olefin gas produced to the mass of the plastic feedstock.

Aspect 41. The method of any one of aspects 28-34, wherein the light olefin gas has a product yield of about 40% to about 90%, wherein the product yield is calculated as the ratio of the mass of light olefin gas produced to the mass of the plastic feedstock.

Aspect 42. The method of any one of aspects 28-41, wherein the light olefin gas comprises a propylene gas.

Aspect 43. The method of any one of aspects 1-42, wherein the catalyst comprises the metal nanoparticle disposed on the support catalyst at a weight percentage of about 1% to about 6%.

Aspect 44. The method of any one of aspects 1-42, wherein the catalyst comprises the metal nanoparticle disposed on the support catalyst at a weight percentage of about 1% to about 4%.

Aspect 45. The method of any one of aspects 1-44, wherein the support catalyst is selected from Y-type zeolite, a pentasil aluminosilicate zeolite, a mordenite zeolite, a beta zeolite, CeO₂, ZrO₂, and α-Fe₂O₃.

Aspect 46. A system, comprising: a microwave chamber, comprising a catalyst, a microwave inlet, a sample inlet, a gas inlet, and an outlet; a microwave generator, configured to generate microwave radiation and deliver the microwave radiation to the microwave chamber via the microwave inlet; a sample feeder, comprising a plastic feedstock and configured to deliver the plastic feedstock from to the microwave chamber via the sample inlet; a gas delivery device, comprising an inert gas and in gaseous communication with the microwave generator via the gas inlet; and a condenser, in gaseous communication with the microwave chamber via the outlet and configured to collect a gaseous product produced in the microwave chamber.

Aspect 47. The system of aspect 46, wherein the catalyst is an M1/support catalyst; wherein the support catalyst is selected from zeolite, CeO₂, ZrO₂, and α-Fe₂O₃; and wherein M1 is a metal nanoparticle selected from Zr, Ga, Cs, Pt, Pd, Ru, and a combination thereof.

Aspect 48. The system of aspect 47, wherein the support catalyst is selected from Y-type zeolite, a pentasil aluminosilicate zeolite, a mordenite zeolite, a beta zeolite, CeO₂, ZrO₂, and α-Fe₂O₃.

Aspect 49. The system of aspect 47 or aspect 48, wherein the catalyst comprises the metal nanoparticle disposed on the support catalyst at a weight percentage of about 1% to about 6%.

Aspect 50. The system of aspect 47 or aspect 48, wherein the catalyst comprises the metal nanoparticle disposed on the support catalyst at a weight percentage of about 1% to about 4%.

Aspect 51. The system of any one of aspects 46-50, wherein the inert gas is selected from N₂, Ar, CO₂, H₂, He, and a combination thereof

Aspect 52. The system of any one of aspects 46-51, further comprising a pyrometer; wherein the plastic feedstock delivered to the microwave chamber forms a reaction mixture with the catalyst; and wherein the pyrometer is configured to measure the temperature of the reaction mixture.

Aspect 53. The system of any one of aspects 46-52, wherein the system is used to perform the method of any one of aspects 1-45.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Production of Propylene from Polypropylene Waste

A direct microwave catalytic upcycling (DMCU) process offers a transformative and sustainable solution for converting polypropylene (PP) waste into propylene. Unlike conventional heating, microwave heating provides rapid, volumetric energy transfer, improving reaction rates, selectivity, and energy efficiency, especially in endothermic catalytic reactions (Refs. 16 and 17). This method leverages the unique interaction between microwave energy and microwave-sensitive catalysts, addressing the limitations of traditional catalytic pyrolysis, which often produces complex mixtures with low selectivity for valuable chemicals (Refs. 18-20). Integrating advanced microwave-responsive catalyst design with microwave catalysis for the direct conversion of PP waste into propylene offers an efficient and sustainable approach to plastic waste upcycling.

Herein, a direct microwave catalytic upcycling approach is introduced that selectively converts PP waste into high yields of propylene using a microwave-responsive catalyst Ru/α-Fe₂O₃, and its performance is compared to conventional heating methods. The stability of the DMCU system on PP conversion is systematically investigated along with product distribution using real-world PP wastes. The results demonstrate that a maximum propylene yield of 57.2 wt. % is achieved at a relatively low temperature of 400° C. under ambient pressure within 30 minutes. The synergy between the Ru/Fe₂O₃ catalyst and microwave energy aids in maximizing propylene yields, with minimal liquid and solid by-products, outperforming conventional heating approaches. This work highlights the potential of DMCU technology as a scalable and efficient solution for the sustainable management of real-world post-consumer PP waste.

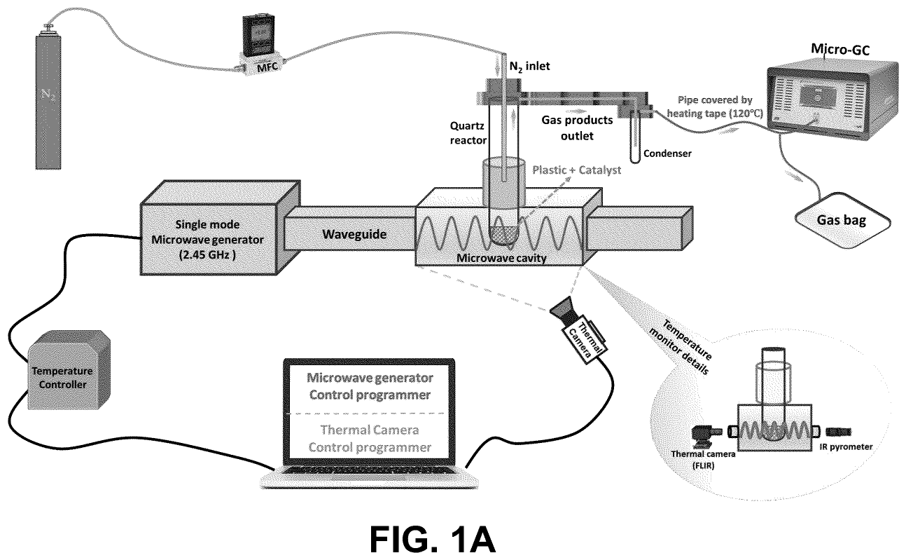

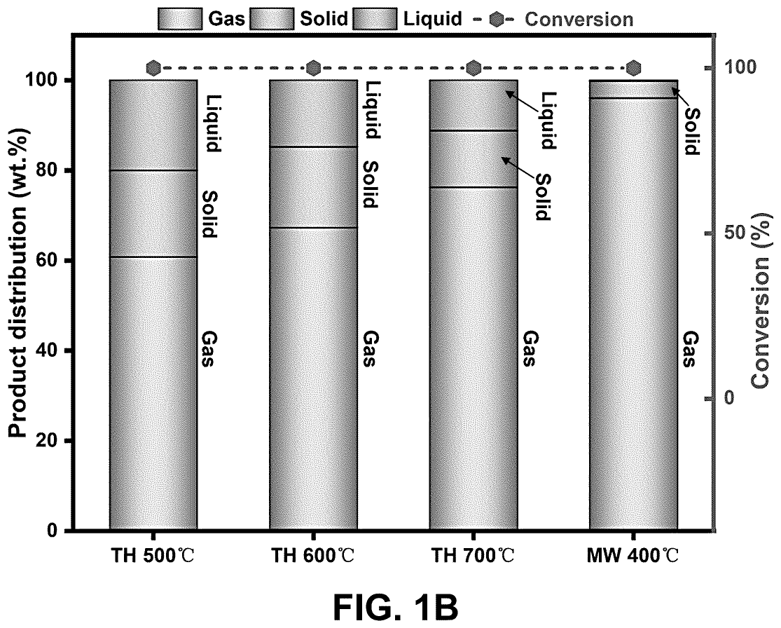

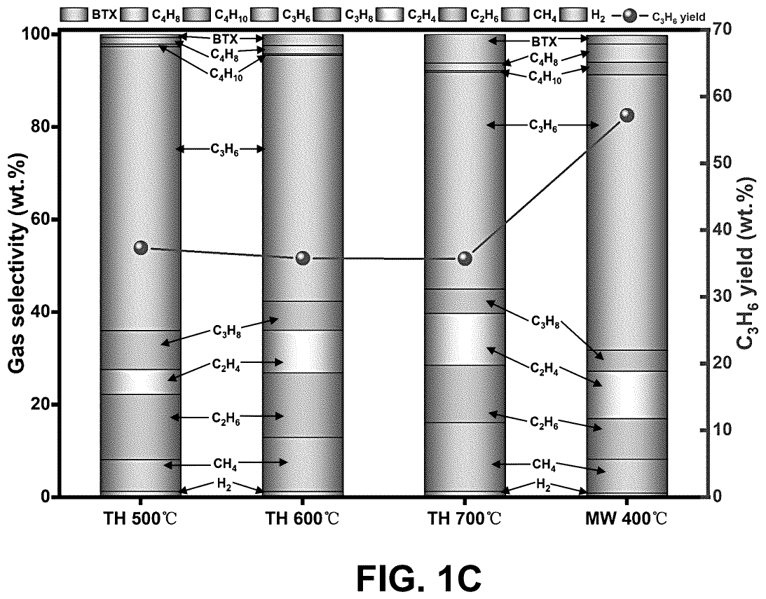

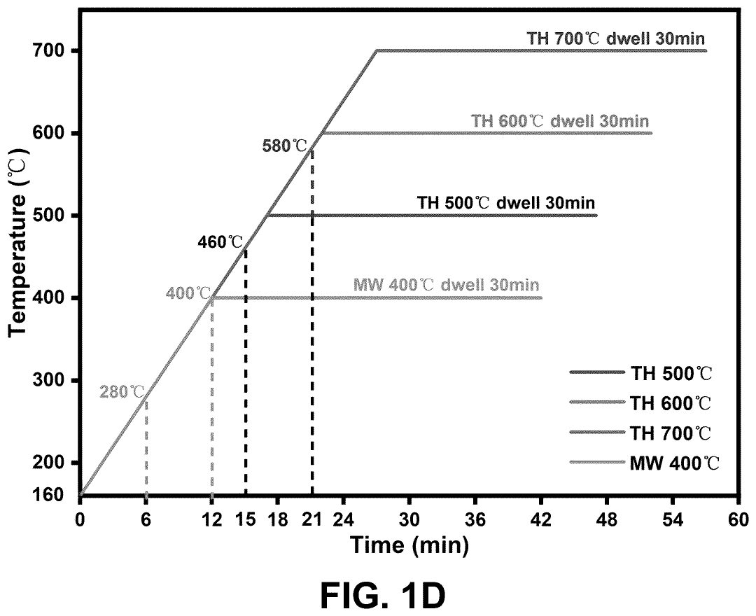

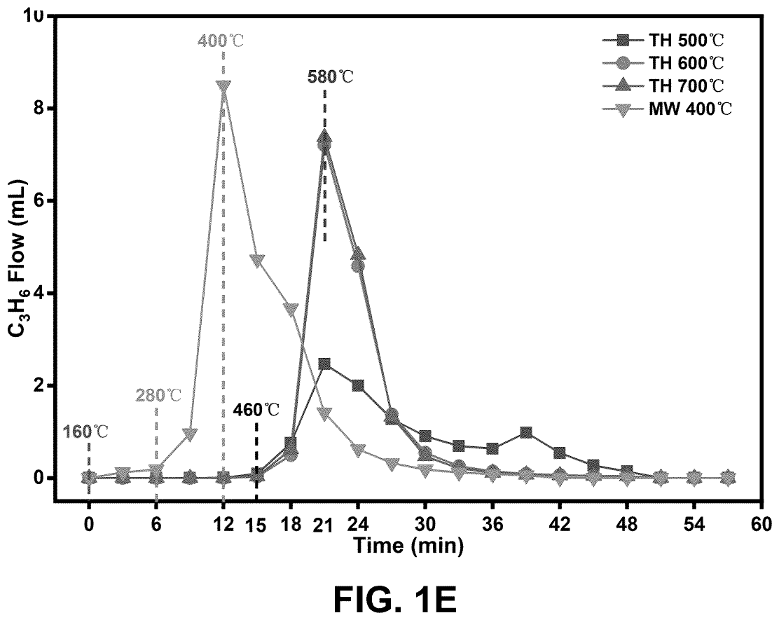

Results and Discussion. Experiments on the direct microwave catalytic upcycling of PP waste were conducted in a batch reactor equipped with a single-mode microwave generator, as depicted in FIG. 1A. For comparison, the upcycling of PP waste was also performed under thermal heating (TH), where all experimental conditions matched those of the MW irradiation group, except for the heating method and reaction temperature. Time on stream (TOS) data was collected at 3-minute intervals, starting at 160° C., with a consistent heating rate of 20° C./min and a dwell time of 30 minutes, as shown in FIG. 1D.

As shown in FIG. 1B, the conversion rates under both MW and TH conditions consistently approach 100%, indicating complete feedstock conversion. However, product distribution analysis reveals a significant advantage for MW heating at 400° C., which yields 96 wt. % gaseous products, with only 3.8 wt. % solid and 0.2 wt. % liquid by-products. This contrasts with TH at 500° C., 600° C., and 700° C., where much higher proportions of liquid and solid by-products are observed. For example, liquid products constitute 20 wt. %, 14.8 wt. %, and 11.2 wt. % at 500° C., 600° C., and 700° C., respectively, while solid products make up 19.2 wt. %, 17.9 wt. %, and 12.6 wt. % at 500° C., 600° C., and 700° C., respectively. These results indicate that even under a relatively lower temperature, MW catalytic upcycling more efficiently cracks PP into gaseous products compared to TH.

FIG. 1C further highlights notable differences in gas selectivity and propylene yield (S_{C3H6} and Y_{C3H6}) between TH and MW heating. Under DMCU at 400° C., propylene selectivity reaches 59.6 wt. %, with a propylene yield of 57.2 wt. %. In comparison, TH achieves its highest propylene selectivity of 61.4 wt. % at 500° C. but with a much lower yield of 37.3 wt. %. At higher TH temperatures (600° C. and 700° C.), both selectivity and yield decline significantly due to increased secondary cracking and by-product formation. Moreover, the DMCU system significantly reduces the formation of less desirable by-products such as CH₄, C₂H₆, and C₃H₈, which are more prominent under TH conditions. The lower production of these alkanes under DMCU suggests that the technology favors selective pathways for propylene formation.

FIG. 1E shows that under microwave (MW) conditions, the C₃H₆ flow rate begins to rise significantly at 280° C., peaking at around 12 minutes when the temperature reaches 400° C., with a peak flow rate exceeding 8 mL/min. This early and pronounced peak demonstrates that DMCU technology efficiently facilitates the catalytic cracking of PP at lower temperatures, rapidly achieving optimal conditions for propylene production. In contrast, TH conditions exhibit delayed propylene production, with distinct differences across the tested temperatures. At TH 500° C., the onset of propylene production is significantly slower, requiring a 3-minute dwell time to reach a modest peak, indicating less effective catalytic activation. At TH 600° C. and 700° C., the peak C₃H₆ flow occurs between 460° C. and 580° C., reflecting the need for higher temperatures to approach the production levels achieved at MW 400° C. Even at 700° C., the peak flow rates under TH remain lower than those observed under MW, underscoring the reduced energy efficiency and slower catalytic activation associated with traditional thermal methods. These findings highlight the advantages of DMCU technology in promoting rapid, efficient, and selective propylene production, achieving higher yields at lower temperatures than conventional thermal methods.

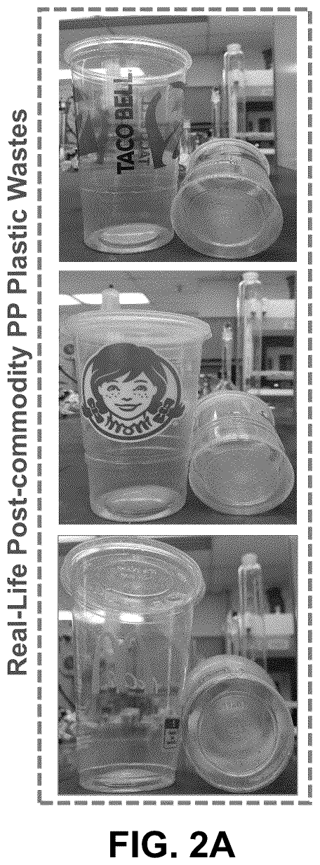

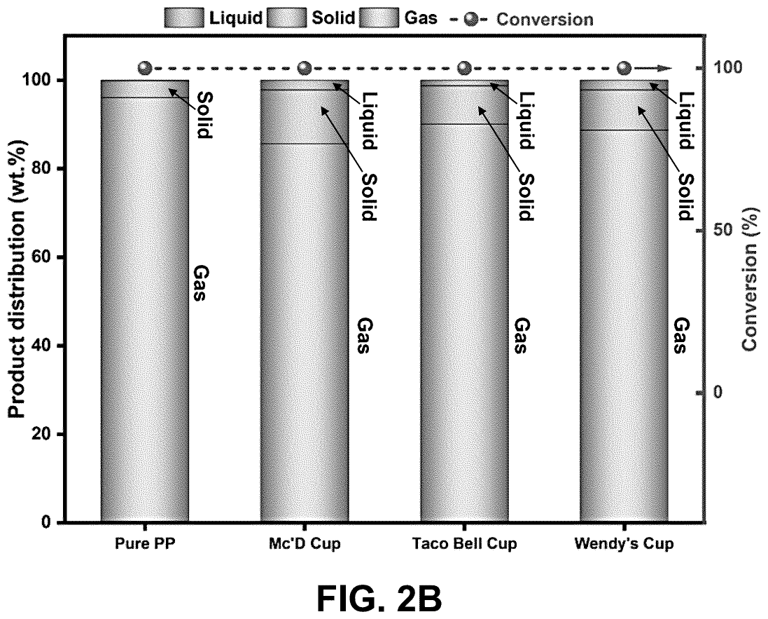

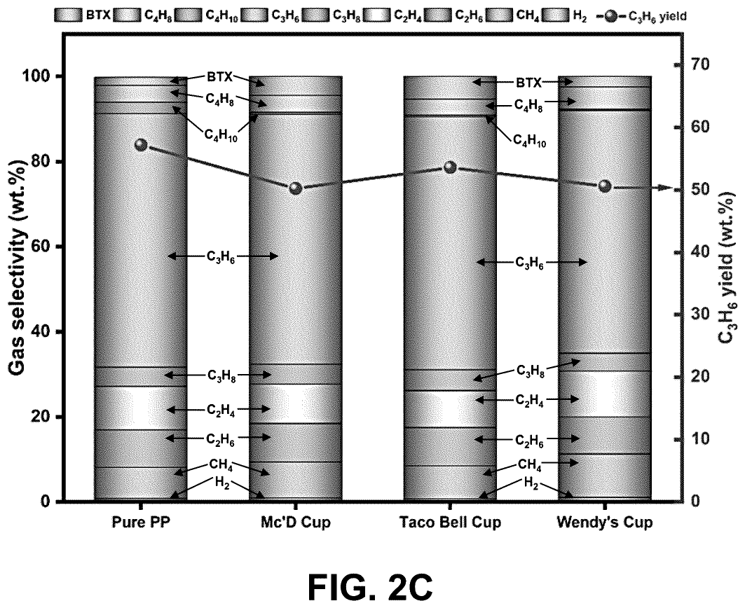

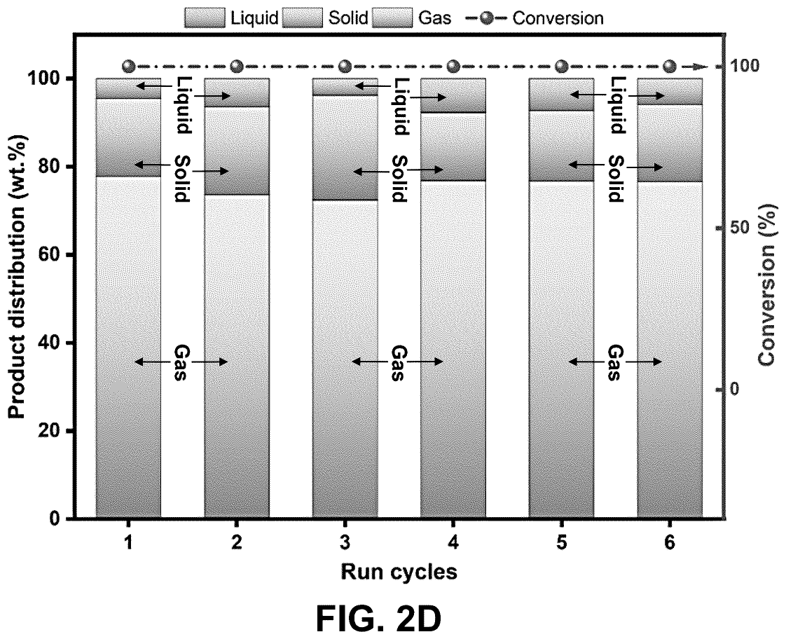

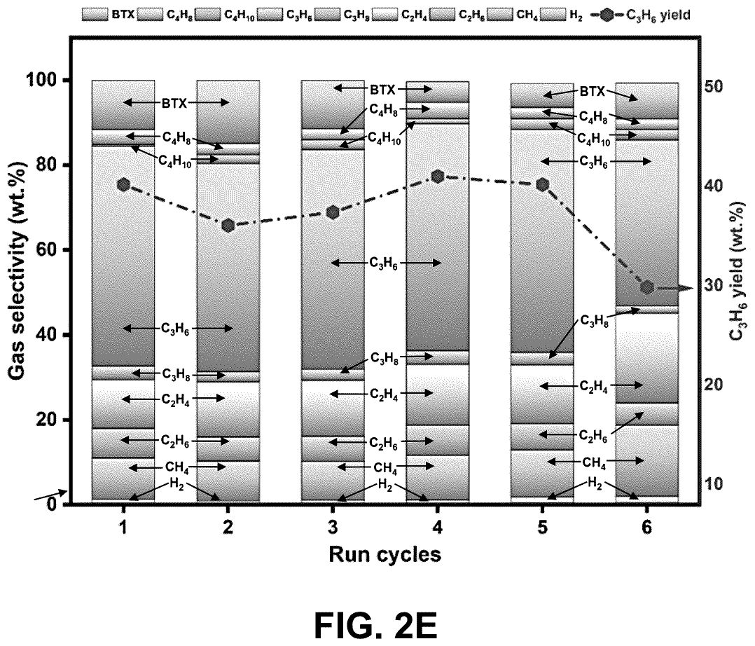

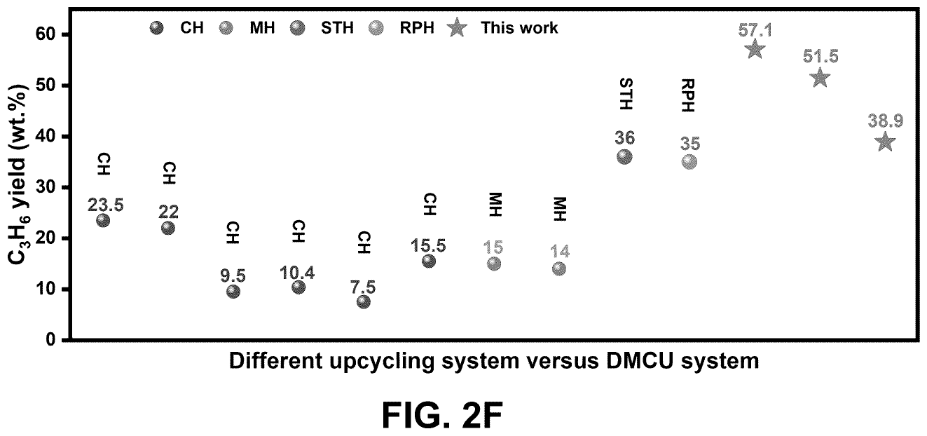

DMCU of Single-use Post-consumer Polypropylene Wastes. Real-world plastics often contain additives and impurities that can hinder catalyst efficacy and affect product distribution (Ref. 14). To evaluate the catalytic performance of the DMCU system with real-world waste, we used single-use PP cups from major fast-food brands, including McDonald's, Taco Bell, and Wendy's (FIG. 2A). The PP cup waste was mechanically ground into a fine powder (≤2 mm) without any pretreatment.

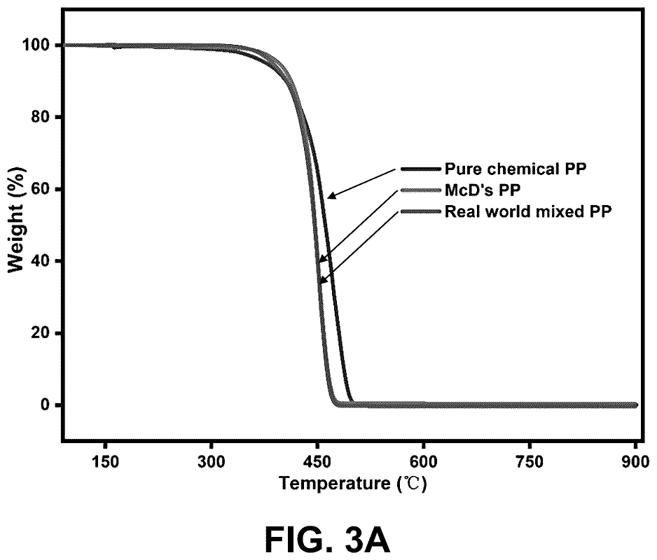

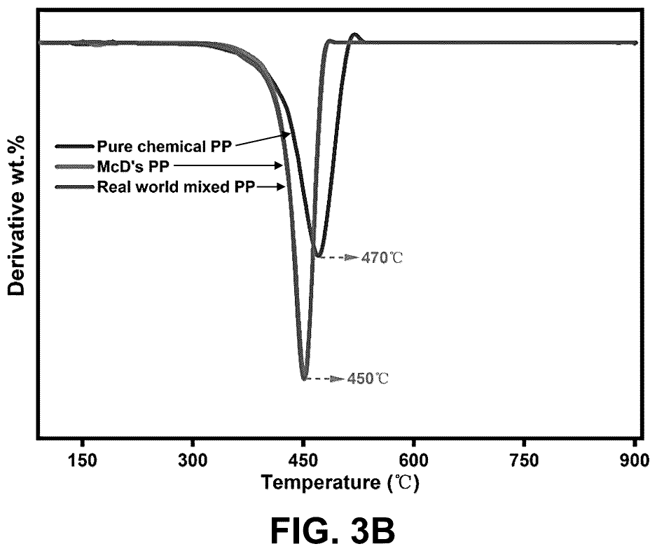

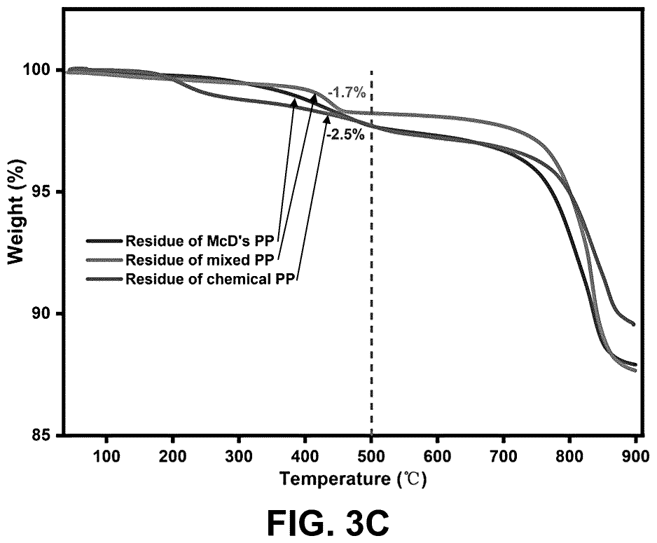

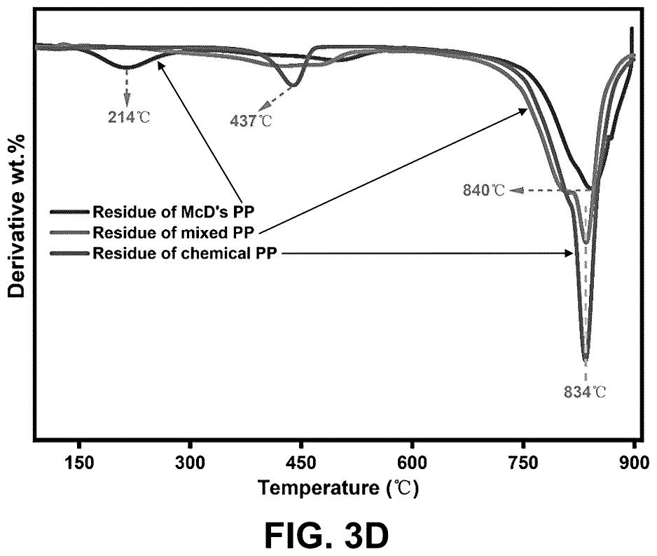

FIG. 2B illustrates the high efficacy of the DMCU system for both pure PP and post-consumer PP cup waste. The near 100% conversion observed in both cases (as confirmed by TGA analysis in FIG. 3C highlights the robustness and broad applicability of this catalytic system for practical waste management. However, the gas product distribution for pure chemical PP is slightly higher than that of post-consumer PP cup waste, suggesting that additives or contaminants in real-world waste slightly influence product distribution, leading to a higher proportion of non-gaseous products (solid and liquid).

FIG. 2C shows consistent propylene selectivity across various PP sources, with gas selectivity of 59.6 wt. % for pure chemical PP and similarly high values for McDonald's (58.7 wt. %), Taco Bell (59.4 wt. %), and Wendy's (57.1 wt. %) cups. This consistency suggests that despite compositional differences, the DMCU system maintains high propylene selectivity. These results indicate that the catalytic system is not only effective for pure chemical PP but also well-suited for upcycling post-consumer single-use PP cups commonly used in fast-food establishments.

To assess the stability of the DMCU system, multiple reaction cycles were conducted using the same batch of catalyst and a mixture of particles from various single-use PP cups. After each cycle, 0.5 g of fresh mixture particles was added to the tube, mixed with the remaining residue, and the reaction was repeated under identical conditions. FIG. 2D shows that the gas product distribution remained consistent across six cycles, with the gas yield stabilizing around 75%, approximately 12% lower than that of single PP cups. This reduction in gas yield is likely due to the additives and contaminants in real-world PP waste, which may resist catalytic cracking. Despite this, the system demonstrated excellent stability, maintaining near-complete conversion across all cycles, underscoring its durability and suitability for processing mixed plastic waste.

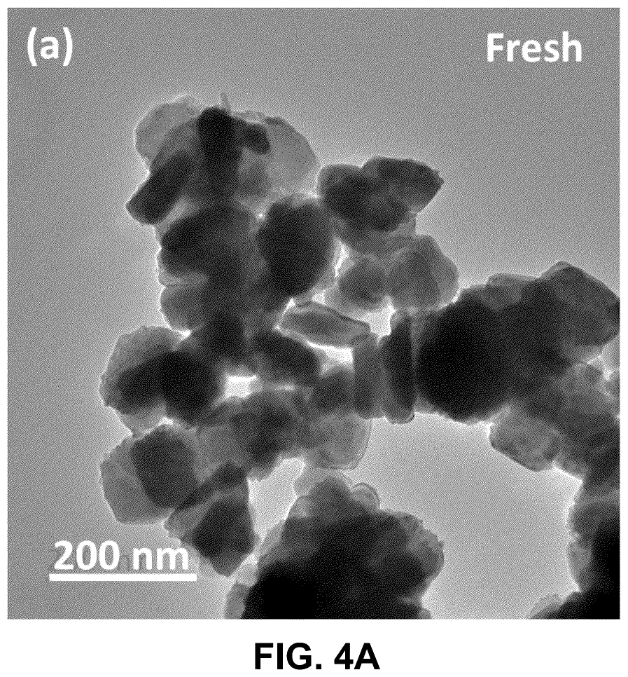

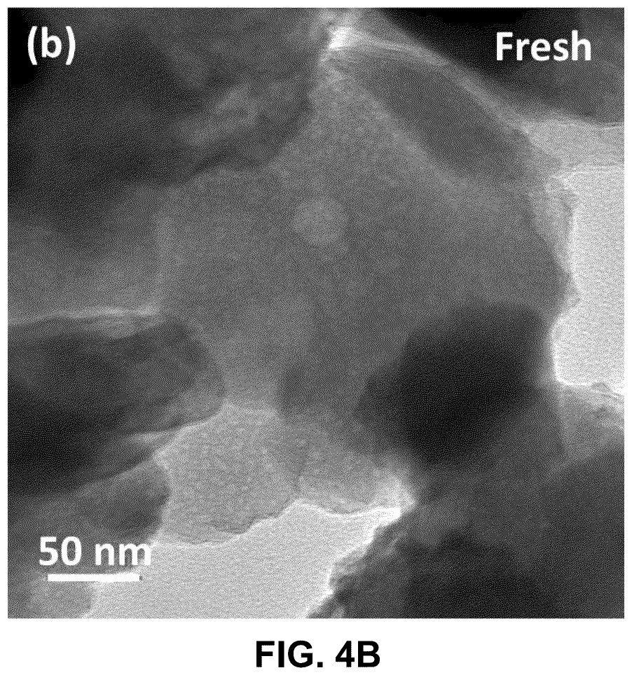

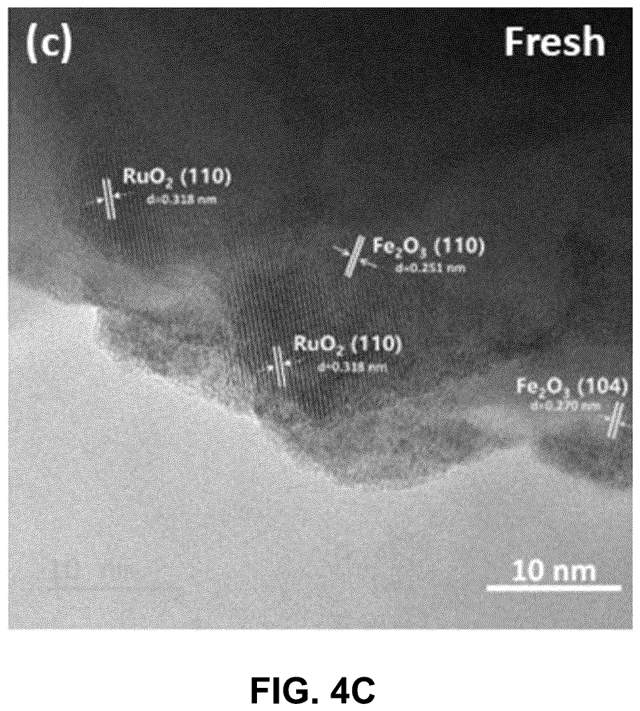

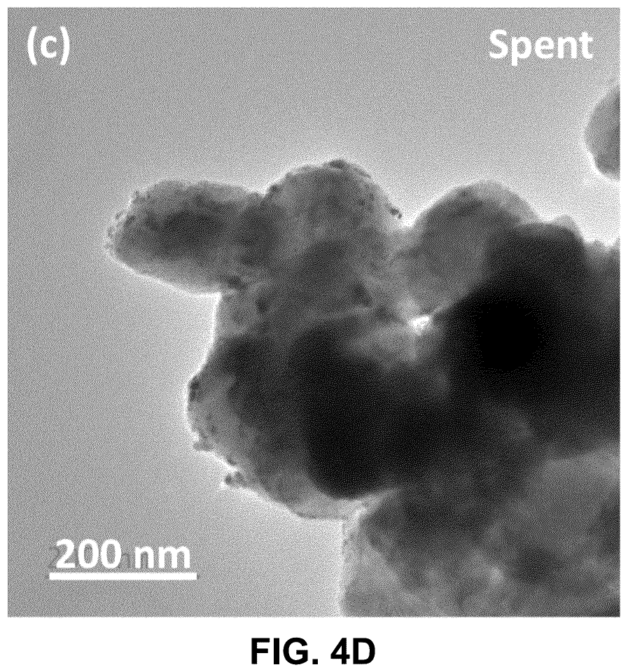

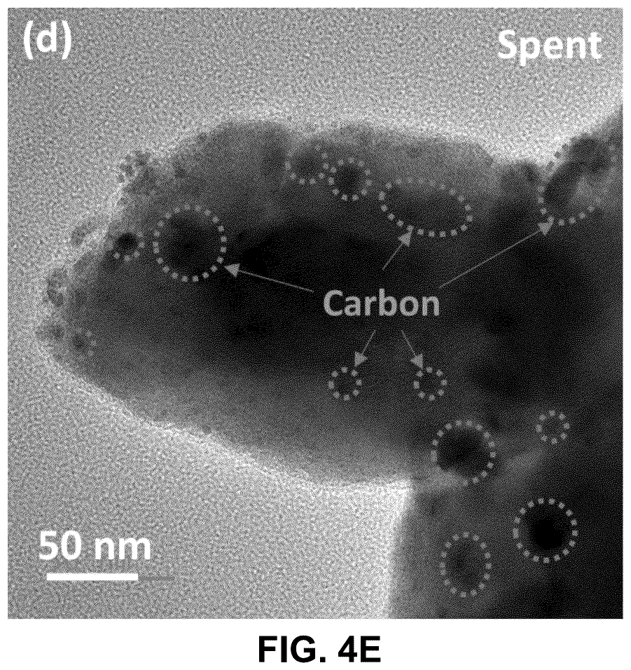

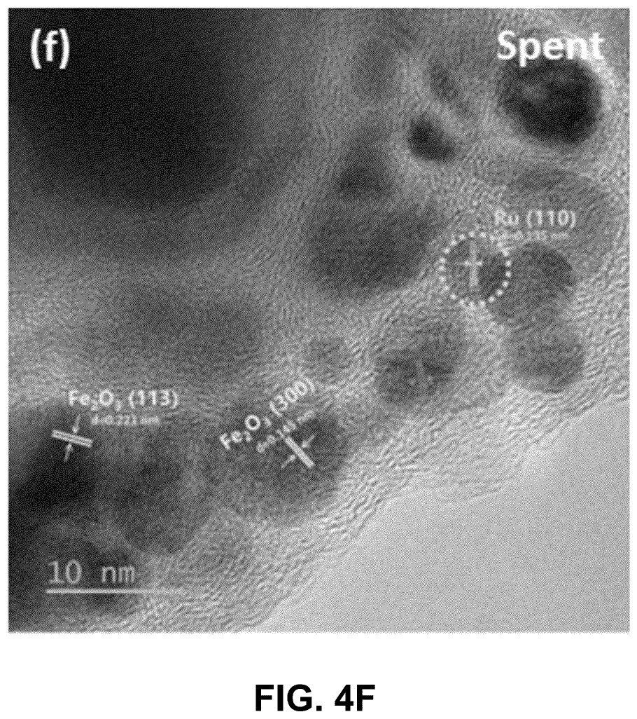

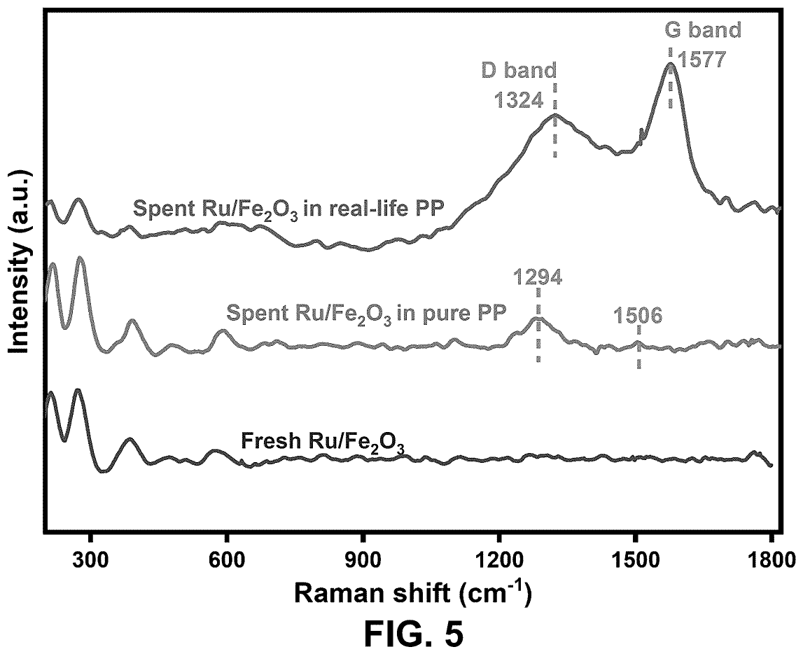

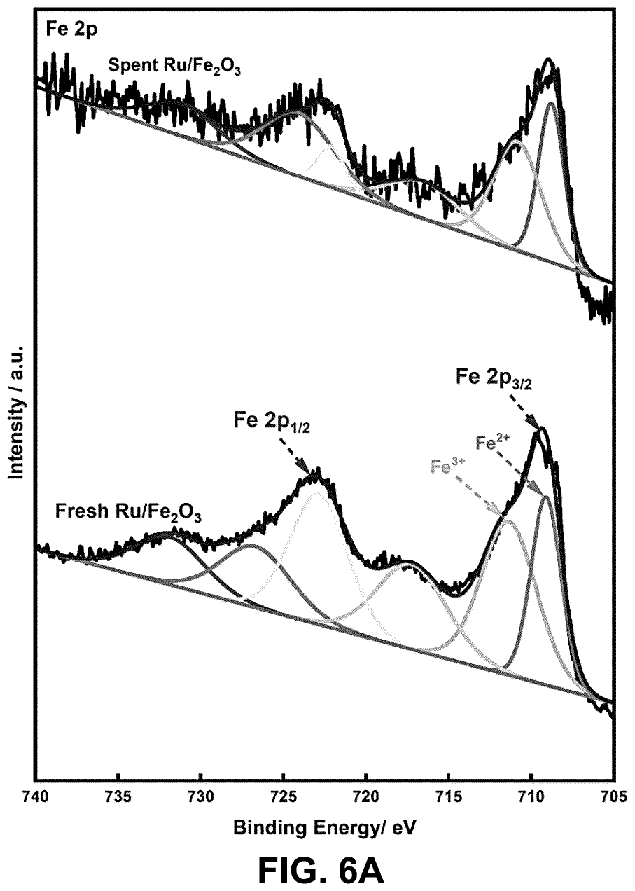

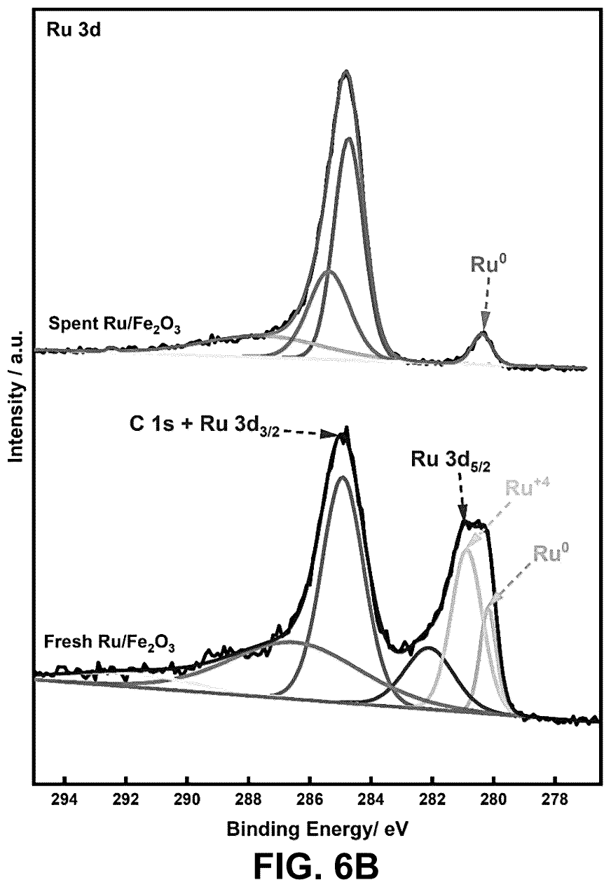

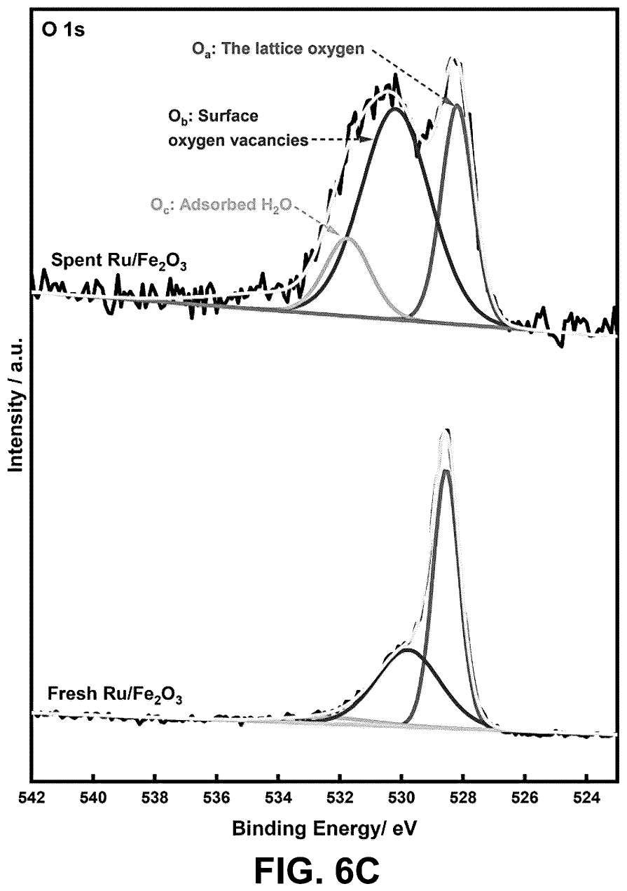

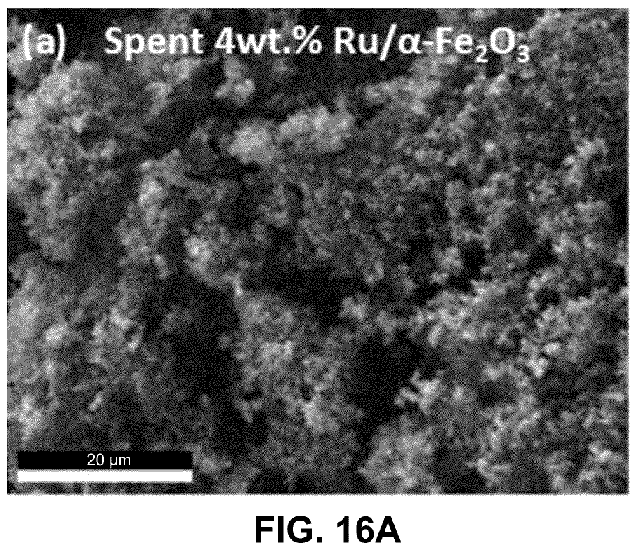

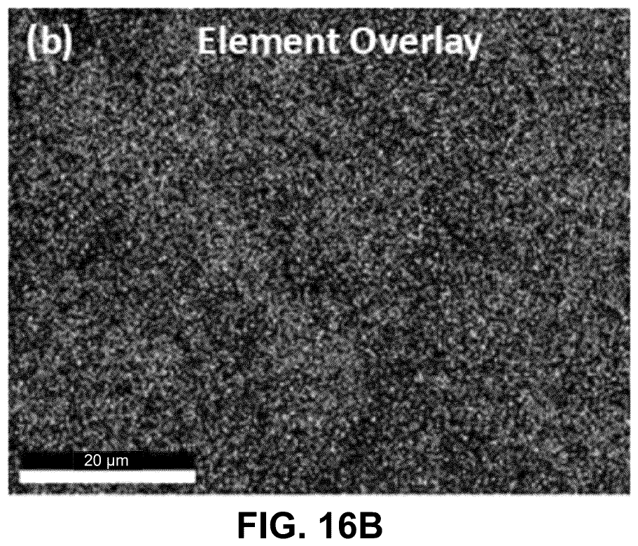

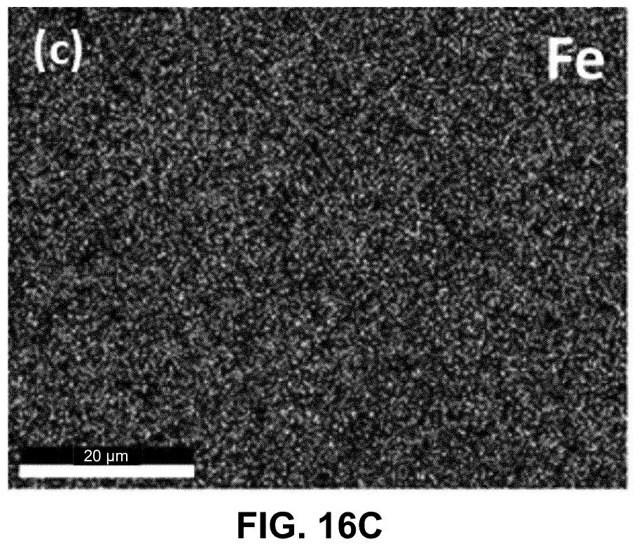

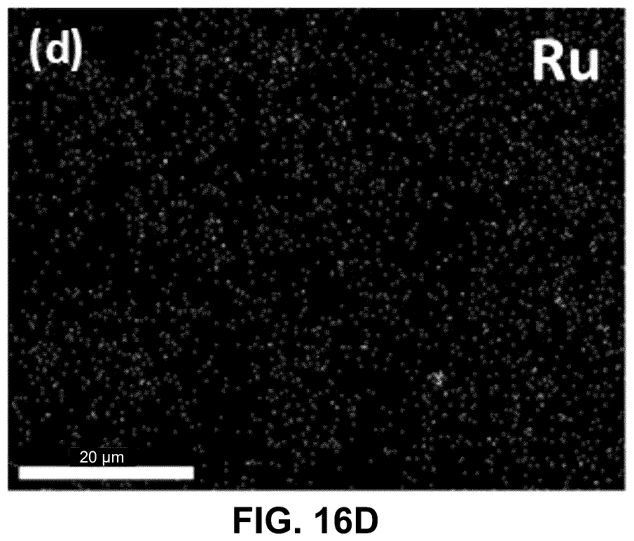

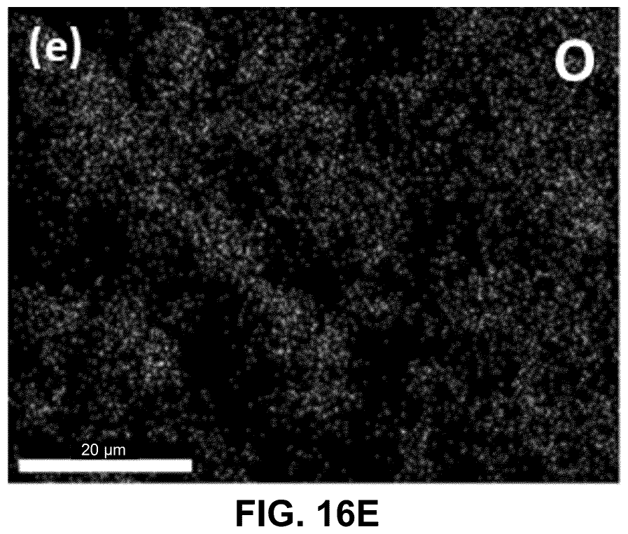

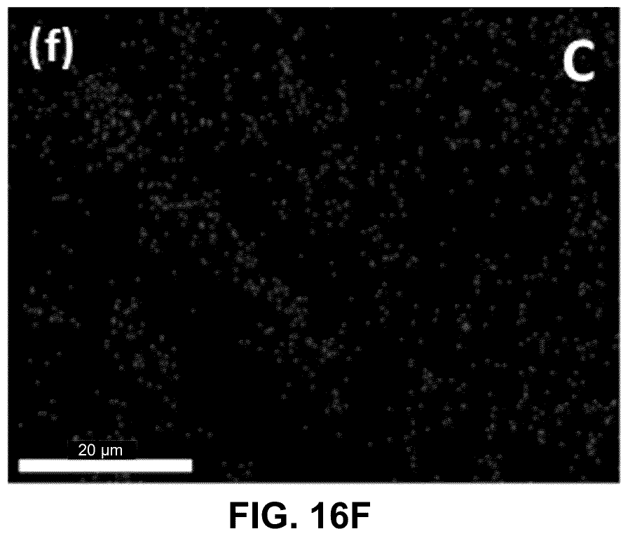

In addition, FIG. 2E reveals that propylene selectivity remained high and stable over the first five cycles, with values of 51.7 wt. %, 49.1 wt. %, 51.7 wt. %, 53.4 wt. %, and 52.4 wt. % for cycles 1-5, respectively. This consistency indicates that the catalytic system maintains high selectivity for propylene, even when processing mixed PP waste. However, in the sixth cycle, propylene selectivity dropped to 39%, while the selectivity for ethylene, methane, and hydrogen nearly doubled compared to the average of the first five cycles. This shift can be attributed to the accumulation of surface carbon on the spent catalyst (as shown in TGA FIG. 3C, TEM FIGS. 4C-4E, SEM-EDX FIG. 16F, and Raman FIG. 5), which enhances microwave absorption and generates localized heating (Ref. 22), thereby promoting deeper cracking of propylene. Additionally, the significant increase in oxygen vacancies (Oᵥ) and the dominance of Ru⁰ species in the spent catalyst (as shown in FIGS. 6B-6C XPS analysis) further enhance dehydrogenation reactions, leading to higher yields of lighter hydrocarbons and gases (Ref. 23). Moreover, the yield of C₃H₆ monomer achieved by DMCU in this Example ranks among the highest compared to published literature using various processes (typically ≤36%; as shown in FIG. 2F and Table 1) (Refs. 13, 14, and 24-31).

In conclusion, these findings demonstrate that the DMCU system effectively upcycles real-world post-consumer PP waste while maintaining high conversion efficiency and propylene selectivity across multiple cycles. This underscores the potential of this system as a scalable solution for plastic waste upcycling, supporting the advancement of a circular economy.

TABLE 1
C₃H₆ yield from PP depolymerization in the present Example
(using DMCU system) compared to the published literature.

| | Reaction | | | C₃H₆ | |
|---|---|---|---|---|---|
| Heating | Temperature | | Carrier | Yield | |
| method | (° C.) | Catalyst | gas | (wt. %) | Refs. |
| CH | 520 | HZSM-5 | N₂ | 23.5 | (Ref. 60) |
| CH | 425 | HZSM-5 | N₂ | 22 | (Ref. 25) |
| CH | 450 | Zeolite Y | N₂ | 9.5 | (Ref. 26) |
| CH | 490 | — | N₂ | 10.4 | (Ref. 27) |
| CH | 600 | Silica gel | Vacuum | 7.5 | (Ref. 28) |
| CH | 668 | — | N₂ | 15.5 | (Ref. 29) |
| Electrified | 600 | — | Ar | 36 | (Ref. 13) |
| STH | | | | | |
| RPH | 750 | H-ZSM-5 | He | ~35 | (Ref. 14) |
| MH | 620 | SiC balls | Vapor | ~15 | (Ref. 30) |
| | | | stream | | |
| MH | 700-900 | SiC balls | N₂ | ~14 | (Ref. 31) |
| DMCU | 400 | Ru/α-Fe₂O₃ | N₂ | 57.2 ^{a} | This |
| DMCU | 400 | Ru/α-Fe₂O₃ | N₂ | 51.5 ^{b} | work |
| DMCU | 400 | Ru/α-Fe₂O₃ | N₂ | 38.9 ^{c} | |

*CH: Conventional heating
*STH: Spatiotemporal heating
*RPH: Rapid Pulse Joule Heating
*MH: Microwave heating
*DMCU: Direct Microwave Catalytic Upcycling,
^{a} for pure PP
^{b} for single-use PP cups
^{c} for the mixed PP cups

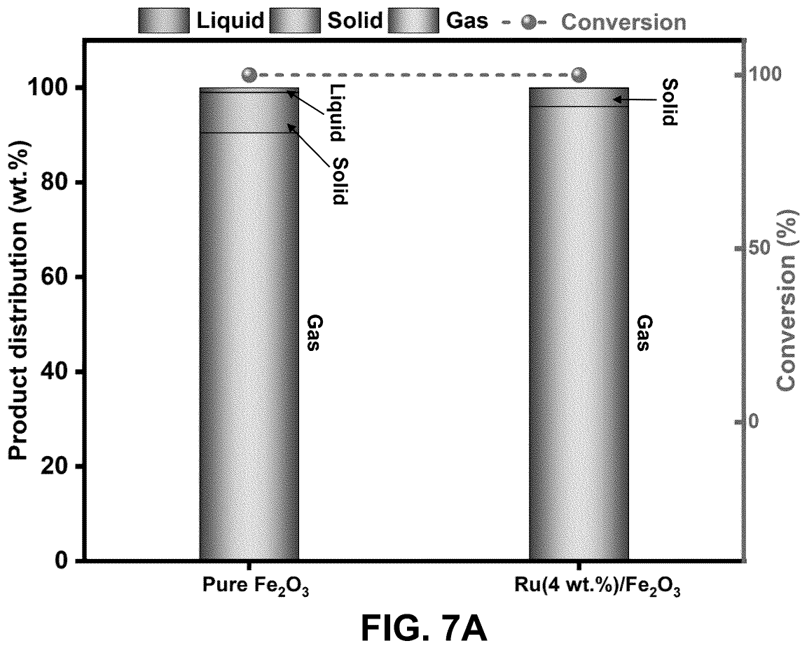

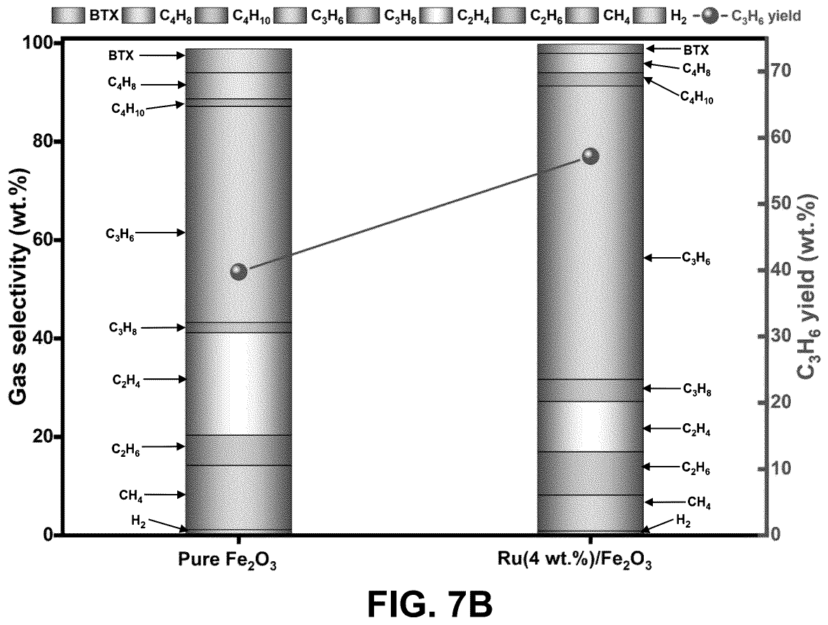

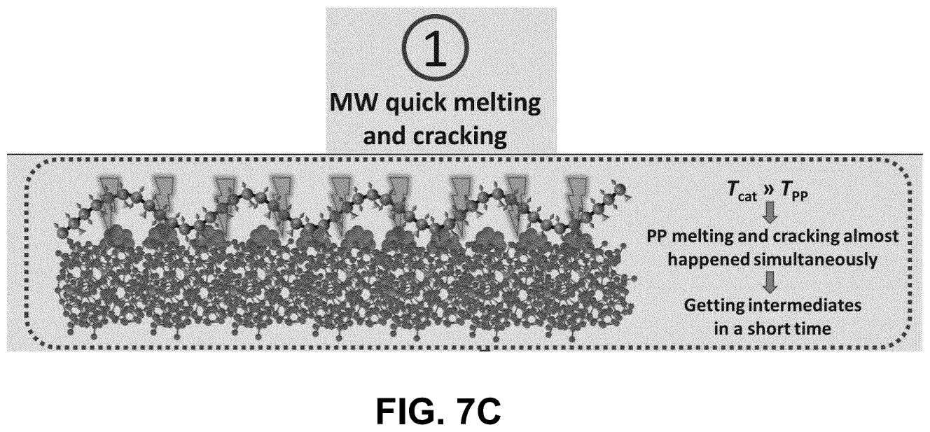

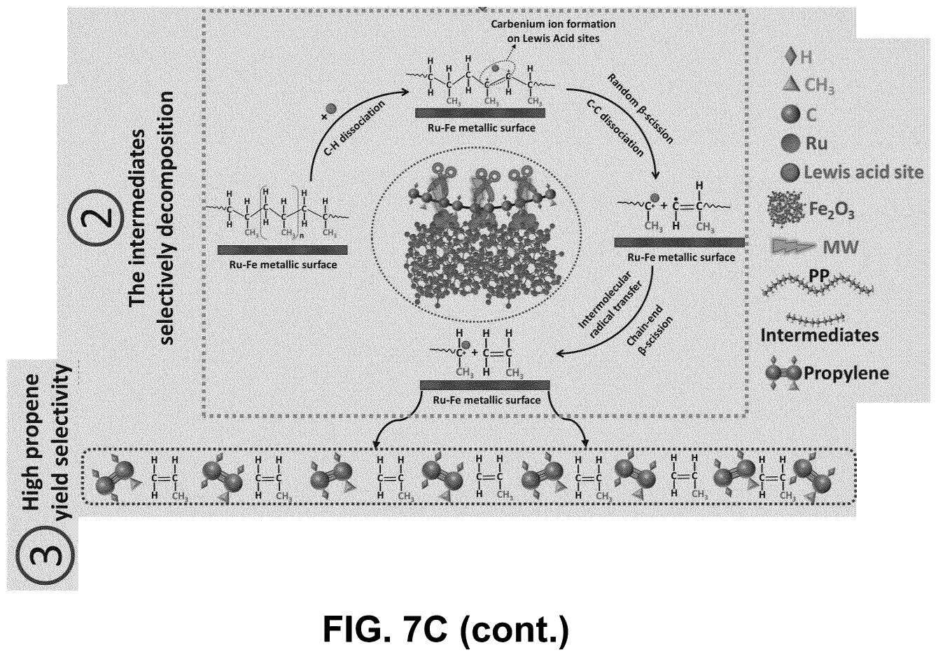

Mechanistic Insights. The selective catalytic upcycling of PP into propylene under microwave irradiation using a microwave-sensitive Ru/α-Fe₂O₃ catalyst is optimized by the synergistic effects of microwave energy, the α-Fe₂O₃ support, and Ru, as illustrated in FIG. 7C.

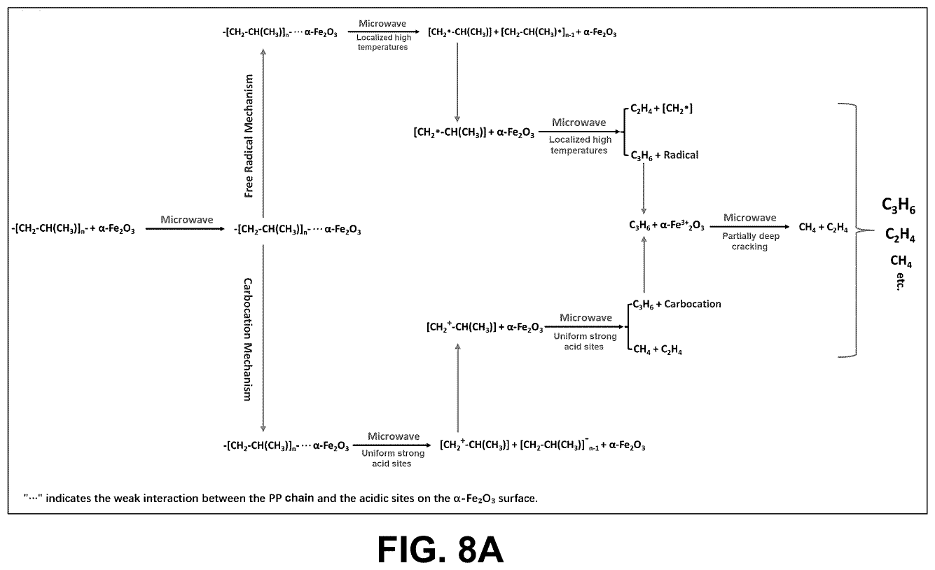

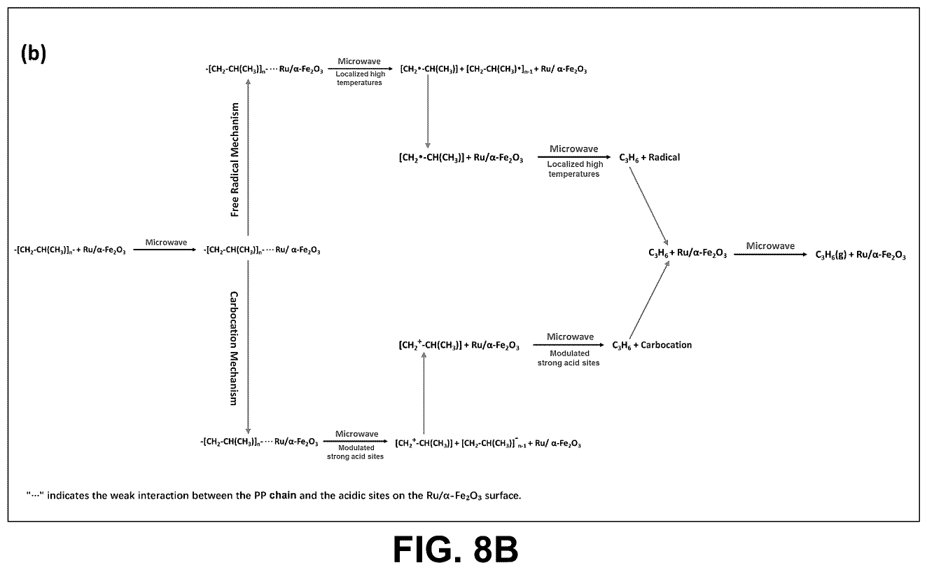

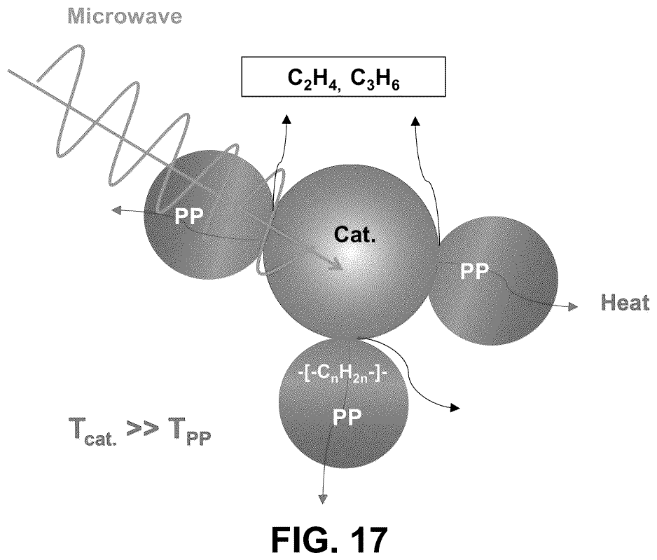

The Role of Microwave Irradiation. Microwave irradiation selectively heats the microwave-responsive Ru/α-Fe₂O₃ catalyst, generating localized high-temperature zones on the catalyst surface (as shown in FIG. 7C and FIG. 8B), which promotes the free radical mechanism in the catalytic upcycling of PP. This selective heating creates a permanent temperature gradient (AT) between the catalyst and surrounding plastic (FIG. 17), focusing energy transfer at the catalyst-polymer interface, suppressing random decomposition, and avoiding non-parasitic heating (Refs. 18, 34, and 61). Free radicals, once formed, are more likely to participate in reactions that result in double bond formation, favoring the production of light olefins such as propylene and ethylene (Ref. 32). The formation of alkanes through the free radical pathway typically requires further radical recombination, which is less likely to occur under the rapid, high-temperature conditions provided by microwave heating, as these conditions favor β-scission over other cracking pathways (Refs. 32 and 33).

At high MW-induced temperatures, free radicals favor β-scission, producing light olefins like propylene and ethylene, while minimizing recombination into alkanes (Refs. 32 and 33). Rapid and selective MW heating reduces residence time and suppresses secondary reactions such as Diels-Alder cycloadditions and hydro-pyrolysis, ensuring higher light olefin selectivity (Ref. 33).

Additionally, the rapid and selective nature of microwave heating significantly shortens the overall residence time, reducing the likelihood of secondary reactions, such as Diels-Alder cycloadditions and hydro-pyrolysis, that could lead to by-product formation (Ref. 33). For example, the reformation of radicals into alkanes requires specific conditions and sufficient time, which are minimized under microwave conditions (Ref. 32). As a result, the free radical pathway with a short residence time predominantly leads to light olefins formation with minimal by-product generation. This efficient energy transfer is further enhanced by the interaction of microwaves with the microwave-sensitive catalyst surface, which directly activates the active sites and significantly boosts catalytic performance (Ref. 34).

MW heating also aligns heat flux with product diffusion (FIG. 17), contrasting conventional methods where heat transfer and mass diffusion are counterproductive. This alignment accelerates product desorption and improves reaction efficiency by enhancing both reaction kinetics and mass transfer (Refs. 13 and 18). The synergistic effects of selective MW heating and optimized energy and mass fluxes make DMCU system highly effective for PP upcycling into valuable olefins (Ref. 34).

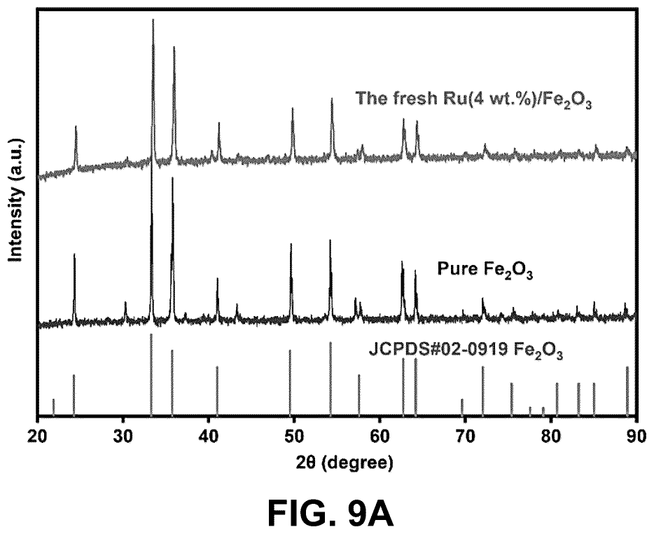

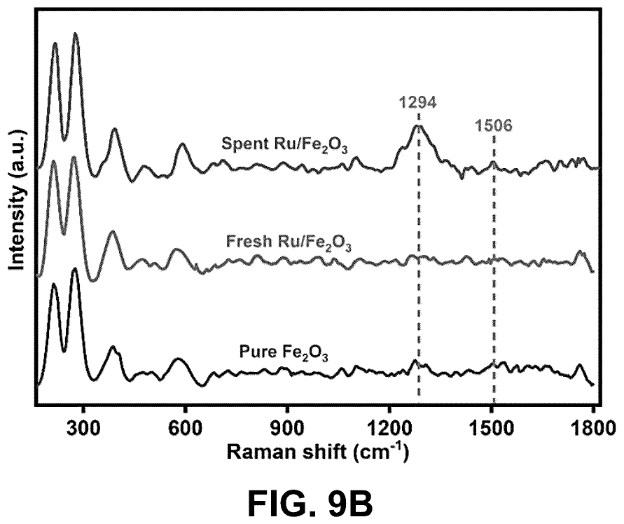

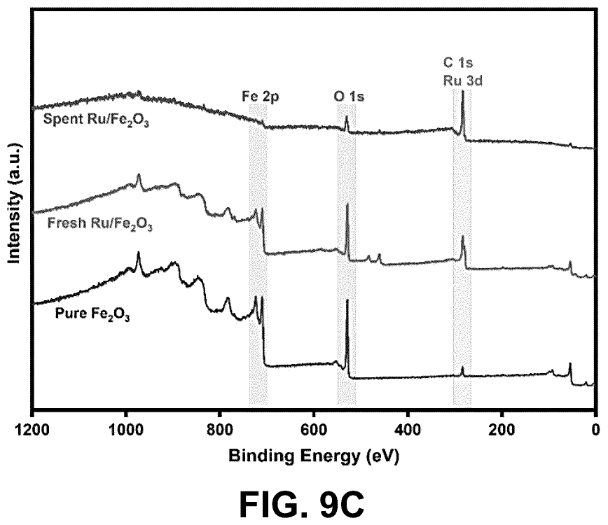

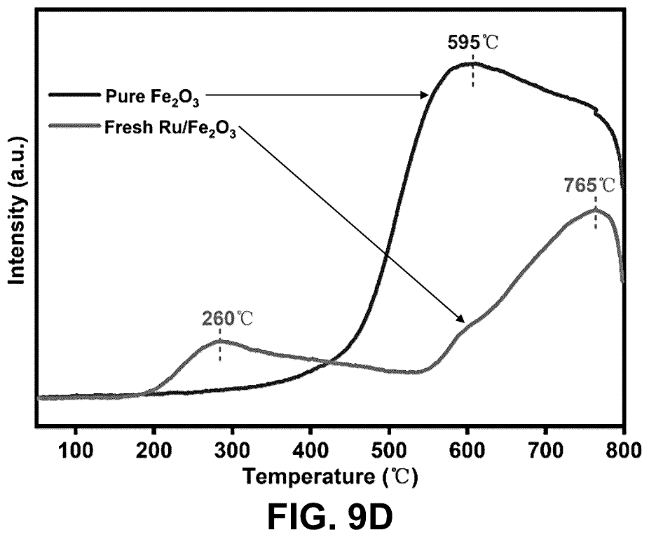

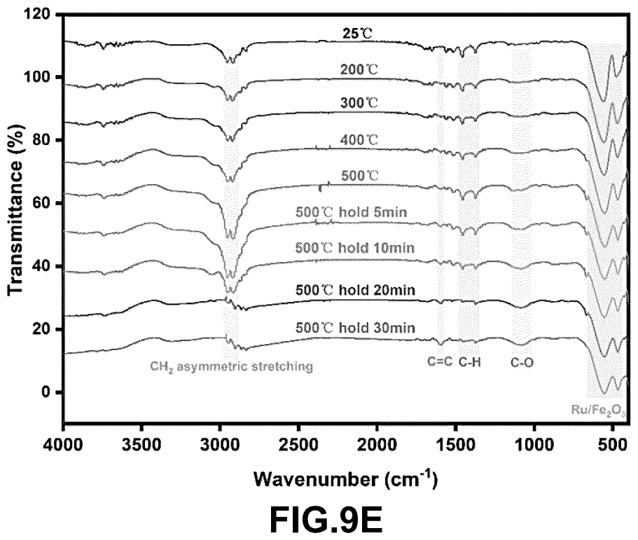

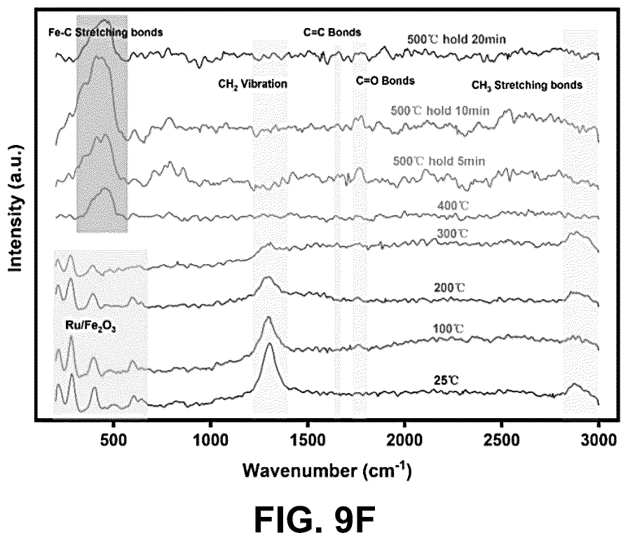

The Role of α-Fe₂O₃. The α-Fe₂O₃ support also plays a role due to its high density of strong acidic sites at high temperature 595° C. (as shown in NH₃-TPD FIG. 9D). In a strong acidic environment, the catalytic upcycling of PP is likely to follow a carbocation mechanism (as shown in FIG. 7C and FIG. 8A) (Refs. 35 and 36).

Specifically, as shown in FIG. 7B, when sole α-Fe₂O₃ is employed, the selectivity for propylene is only 70% of that observed with Ru/α-Fe₂O₃, while the selectivity for methane and ethylene is doubled. These differences arise from strong and relatively uniform acid sites of sole α-Fe₂O₃, promotes propylene deeper cracking, resulting in the formation of smaller hydrocarbons like methane and ethylene (Ref. 37). Also, on sole α-Fe₂O₃ surfaces, the high electron affinity and unpaired electrons of Fe³⁺ enable it to stabilize free radicals, facilitating their further cleavage by either accepting electrons (in the case of radicals), which leads propylene intermediates to further cleavage to the formation of smaller hydrocarbons such as methane and ethylene (as shown in FIG. 8A) (Refs. 38 and 39).

These results suggest that the carbocation mechanism on sole α-Fe₂O₃ is primarily driven by the activation of tertiary carbons in the PP chain, as tertiary carbons are more reactive than primary and secondary carbons in the PP backbone (Refs. 40 and 41). The resulting tertiary carbenium ion intermediates undergo rapid β-scission, leading to the efficient formation of stable light olefins, such as ethylene and propylene (Refs. 14 and 36). The high reactivity of carbocations makes them particularly effective in eliminating adjacent C—H bonds, resulting in the production of light olefins rather than saturated alkanes or hydrogen gas (Ref. 42). Moreover, the thermal stability of α-Fe₂O₃ ensures sustained catalytic activity under high-temperature conditions induced by microwave irradiation, while its effective microwave absorption converts electromagnetic energy into chemical activation energy, driving the reaction efficiently (Ref. 43).

The Role of Ru. The incorporation of Ru onto the α-Fe₂O₃ support enhances the catalyst's activity and selectivity through several interrelated properties. First, Ru's electron-donating and accepting properties modify the electronic structure of the α-Fe₂O₃ surface, enhancing the acidity of the catalytic sites and lowering the activation energy required for the cleavage of C—C bonds in PP (As shown in FIG. 9D NH₃-TPD analysis). This modification facilitates more efficient and selective propylene formation (Ref. 44). Additionally, Ru stabilizes reactive intermediates such as free radicals and carbocations by delocalizing their electron density or charge, reducing their reactivity and preventing recombination reactions that typically lead to by-products like H₂ and alkanes when compared to sole α-Fe₂O₃ (Refs. 33, 45). By maintaining these intermediates in a controlled, less reactive state, Ru guides the reaction pathway towards the desired propylene product, thereby enhancing both the efficiency and selectivity of the catalytic process. Moreover, Ru increases the number of active catalytic Ru—Fe sites on the α-Fe₂O₃ surface (as shown in FIG. 7C and FIGS. 6A-6C XPS analysis), providing more locations for the selective cleavage of PP and further boosting propylene yield. Together, these properties synergistically improve the catalytic performance of Ru/α-Fe₂O₃ catalyst, making Ru an essential component in advancing the efficiency and sustainability of catalytic PP upcycling. In addition, as shown in FIG. 7A, less liquid and solid by-products are observed for the Ru/α-Fe₂O₃ catalyst compared to the sole α-Fe₂O₃ catalyst.

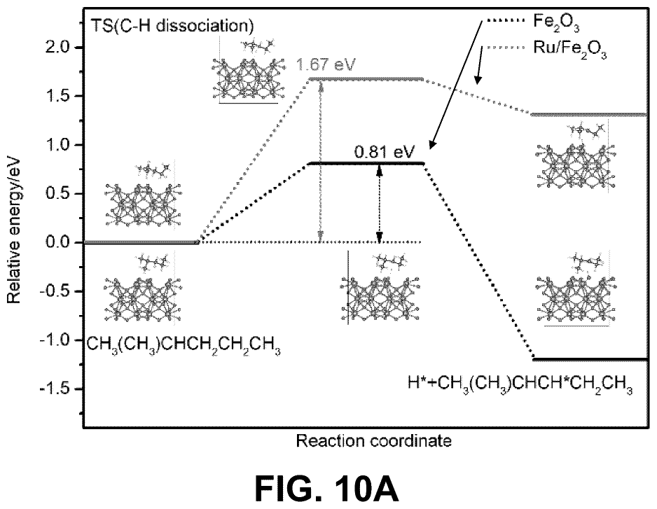

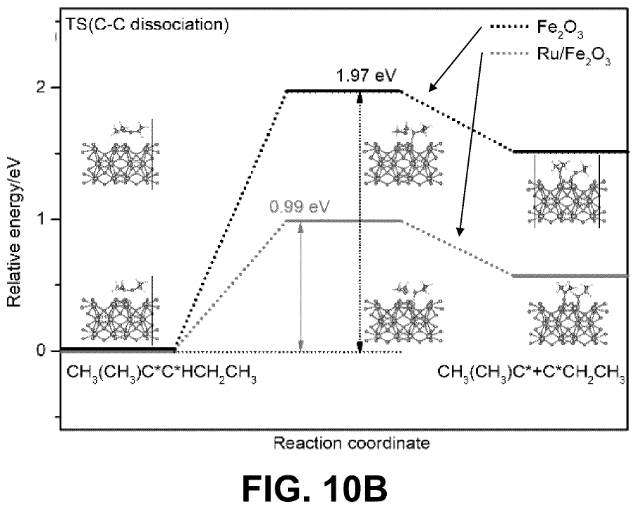

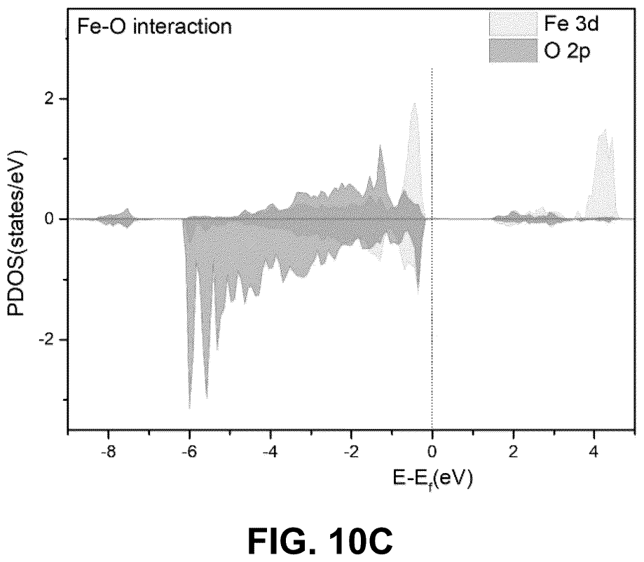

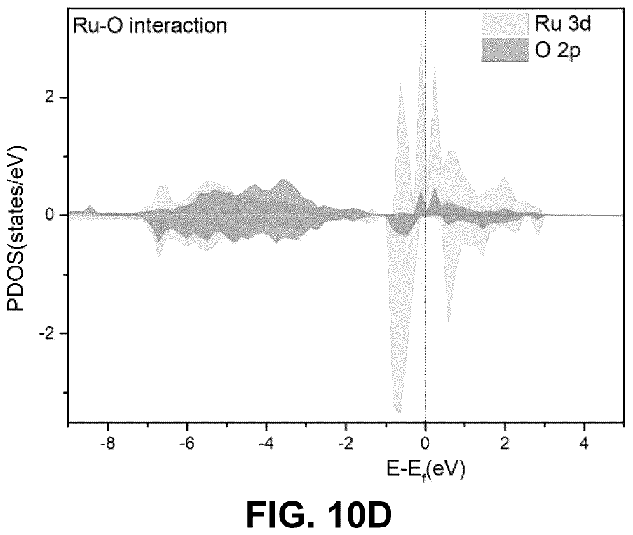

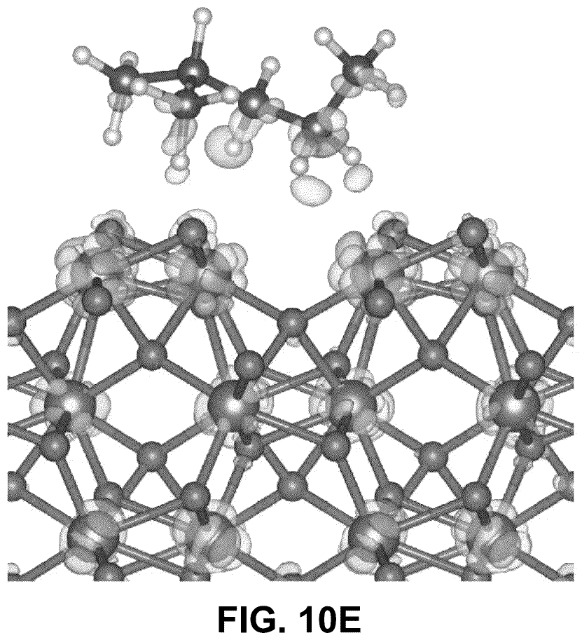

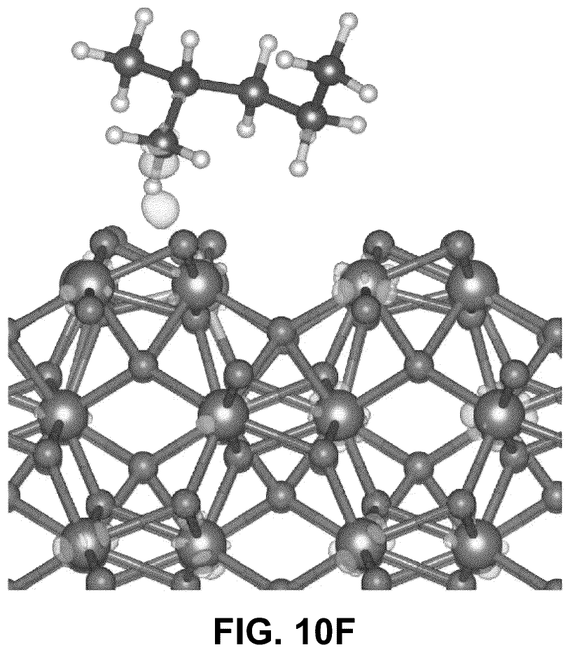

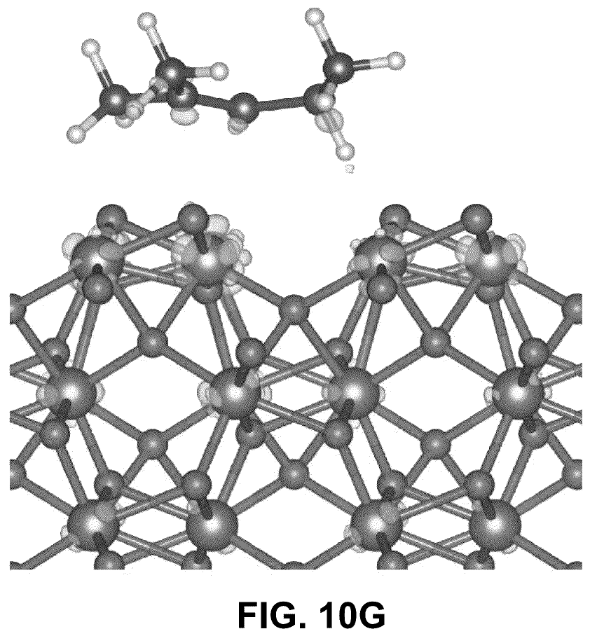

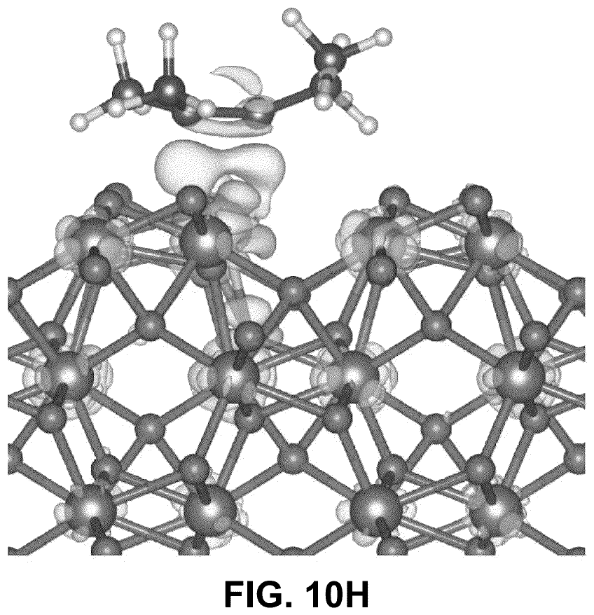

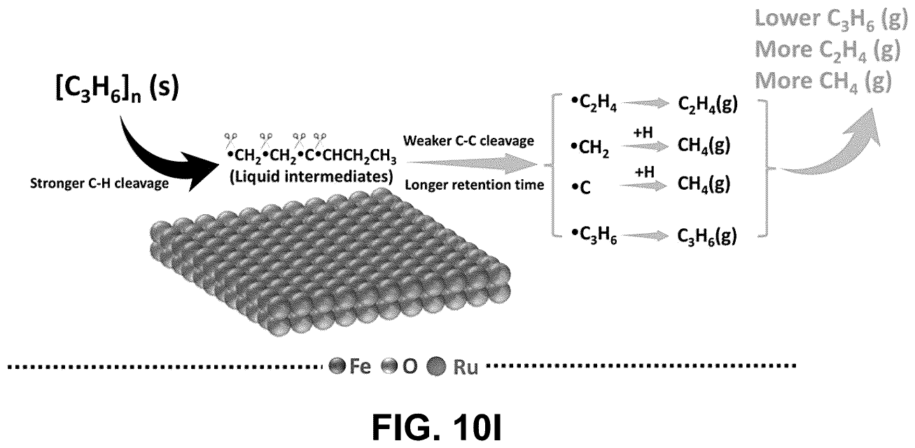

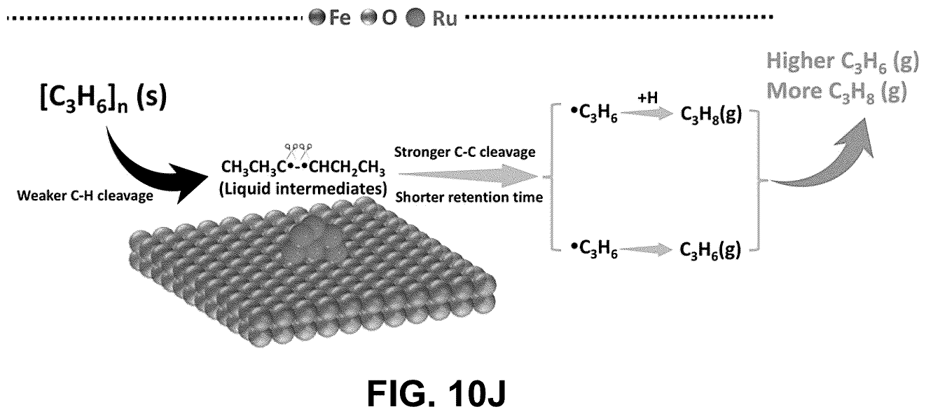

To further clarify the role of Ru in promoting propylene selectivity, detailed DFT calculations were conducted to compare the catalytic behavior of Fe—O and Ru—O active sites in PP upcycling, focusing on the activation of C—H and C—C bonds. As shown in FIG. 10A, Fe—O has a lower energy barrier for C—H bond cleavage (0.81 eV) compared to Ru—O (1.67 eV), indicating its superior ability to activate C—H bonds and promote dehydrogenation in solid-solid reactions (Ref. 46). Conversely, Ru—O favors hydrogenation as the higher C—H dissociation barrier may lead to lower energy barrier for hydrogenating unsaturated carbons, explaining the increased formation of alkanes like propane and ethane in Ru/Fe₂O₃-catalyzed reactions (FIG. 7B).

For C—C bond activation, FIG. 10B reveals that Ru—O has a significantly lower energy barrier (0.99 eV) compared to Fe—O (1.97 eV), enabling more efficient cleavage of long-chain hydrocarbons (intermediates as shown in FIG. 7C). While Fe—O requires multiple active sites to effectively activate C═C bonds after C—H dissociation, slowing the reaction and leading to deeper cracking (methane and ethylene formation), the stronger C═C adsorption of Ru—O allows for efficient C—C bond cleavage even at single sites, leading to faster conversion of long-chain molecules into light olefins like ethylene and propylene.

Furthermore, as shown in FIGS. 10C-10D, near the Fermi energy (0 eV), Ru 5d orbital has a strong overlap with O 2p orbital, while Fe 3d orbital overlaps O 2p orbital at lower energy position, and some O 2p orbital locates in higher energy level, implying a strong Ru—O interaction and weak Fe—O bond. In addition, the calculated d-band center of Fe is −2.3 eV while Ru has a higher d-band center at −1.9 eV, which also accounts for the stronger Ru—O bonding energy and lower ability to activate C—H breakage.

Electron density maps (FIGS. 10E-10H) display the charge transfer before and after simplified saturated and unsaturated long chain molecules (CH₃CH₃CHCH₂CH₂CH₃ and CH₃CH₃CCHCH₂CH₃) adsorbed on sole Fe₂O₃ and Ru/Fe₂O₃. For CH₃CH₃CHCH₂CH₂CH₃, a large number of electrons are transferred from the sole Fe₂O₃ to adsorbed molecule, while for CH₃CH₃CHCH₂CH₃, Ru/Fe₂O₃ donates more electrons to adsorbed molecule. This phenomenon further highlights Fe₂O₃'s stronger affinity for C—H bonds and Ru/Fe₂O₃'s enhanced C—C bond activation.

The combination of faster C—H hydrogenation and more efficient C—C cleavage on Ru—O explains the increased production of propane, ethane, and propylene, aligning with experimental data (FIG. 7B). This mechanistic understanding underscores Ru's superior C—C bond cleavage and hydrogenation properties, driving higher selectivity for propylene, while Fe—O's stronger C—H activation leads to deeper cracking and smaller hydrocarbon formation. These findings complement experimental results, demonstrating the effectiveness of Ru/Fe₂O₃ in propylene production from microwave-assisted PP upcycling.

In summary, the synergy between microwave-responsive Ru/α-Fe₂O₃ catalyst and microwave energy is key to the efficient upcycling of PP into propylene. Microwave irradiation generates localized high-temperature zones, promoting C—C bond cleavage and olefin formation while minimizing secondary reactions. α-Fe₂O₃ activates C—H bonds, leading to dehydrogenation, but also deeper cracking into smaller hydrocarbons. The addition of Ru enhances the catalyst's selectivity by lowering the energy barrier for C—C bond cleavage and stabilizing reactive intermediates, driving propylene formation. DFT calculations support that Ru selectively promotes C—C activation, aligning with experimental results, and demonstrating the catalyst's effectiveness in microwave-assisted PP upcycling.

Chemicals and Catalyst Preparation. Pure chemical PP (MW˜35,000) was purchased from Sigma-Aldrich without further treatment. Real-world PP waste was collected from major fast-food brands, such as McDonald's, Taco Bell, and Wendy's. α-Fe₂O₃, Ruthenium (III) chloride, (Ru 47.7% min, 3.110 g/mL), and Chloroform (CHCl₃) were bought from Sigma-Aldrich.

Microwave-responsive catalyst α-Fe₂O₃ loaded with 4 wt. % Ru (Ru (4 wt. %)/Fe₂O₃) was synthesized using wetness impregnation method. An appropriate amount of Ruthenium (III) chloride was dissolved in 50 mL DI water in the beaker and the solution was ultrasonicated for 10 min. Afterwards, 5 g α-Fe₂O₃ was transferred into the beaker and the mixture was stirred at 400 rpm overnight. Then, the mixture was dried at 100° C. for 6 hours, following calcination at 550° C. for 4 hours. Finally, the catalyst was reduced using pure H₂ at 150° C. for 6 hours with 2° C./min heating rate. Catalysts with target Ru loadings of 2 and 6 wt. % were prepared via the same wetness impregnation-calcination-reduction protocol, using varying quantities of ruthenium (III) chloride to achieve the desired metal loadings.

Experimental Setup and Procedure. As shown in FIG. 1A, the DMCU system was operated using a Sairem microwave control system equipped with a 2.45 GHz solid-state generator, providing a maximum forward power of 1 kW. A TE₁₀ mono-mode cavity acted as the applicator, with closed surfaces of perfect electric conductors. An infrared pyrometer monitored the reactant temperature and regulated the forward microwave power.

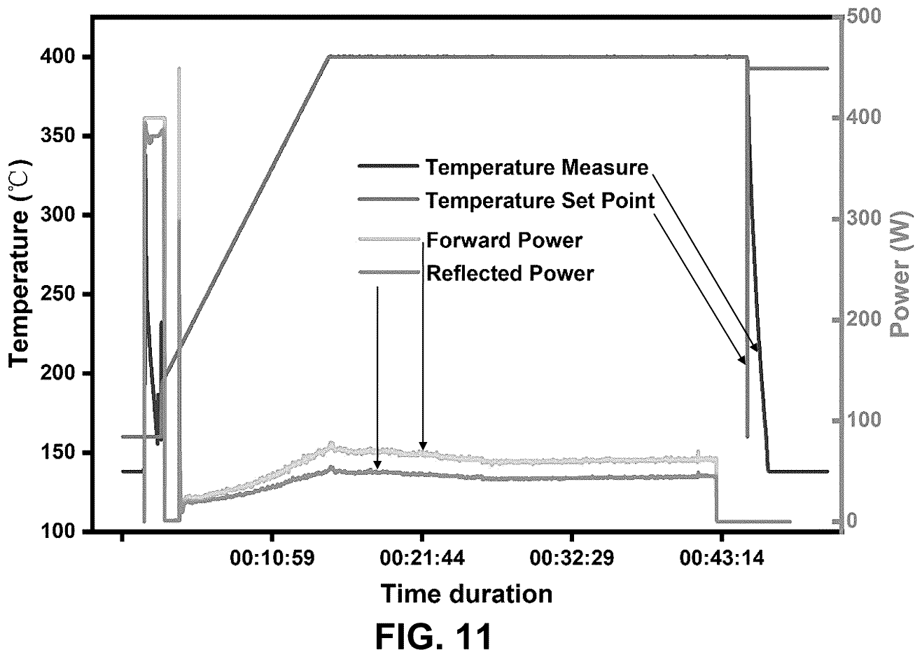

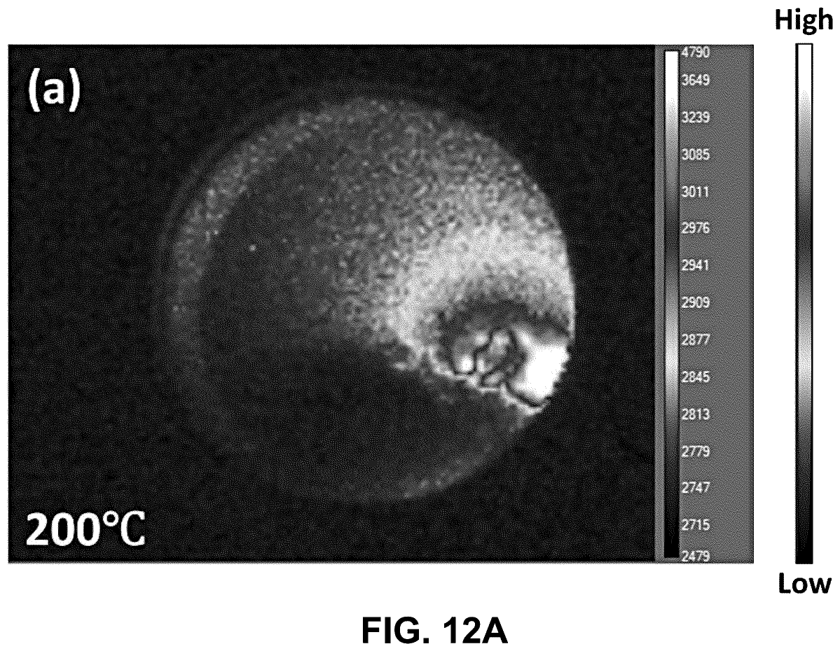

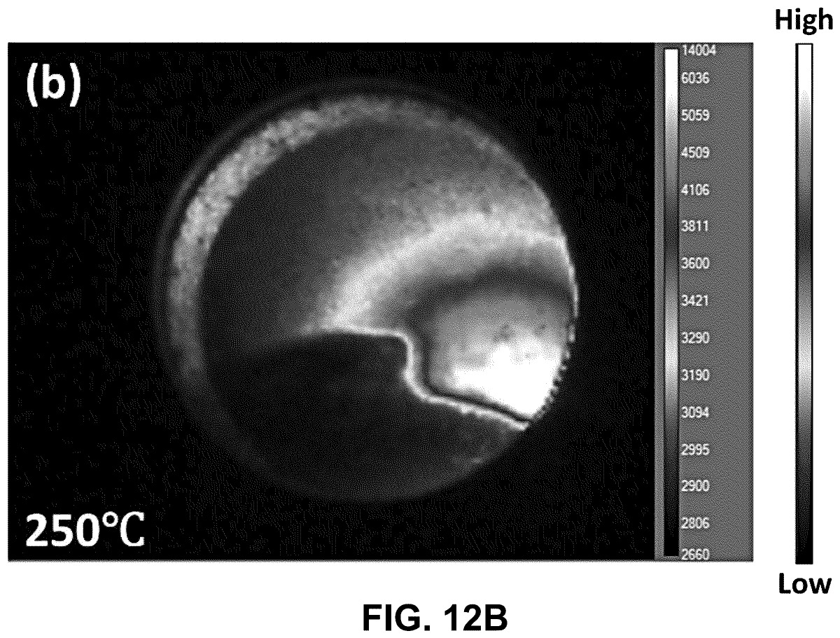

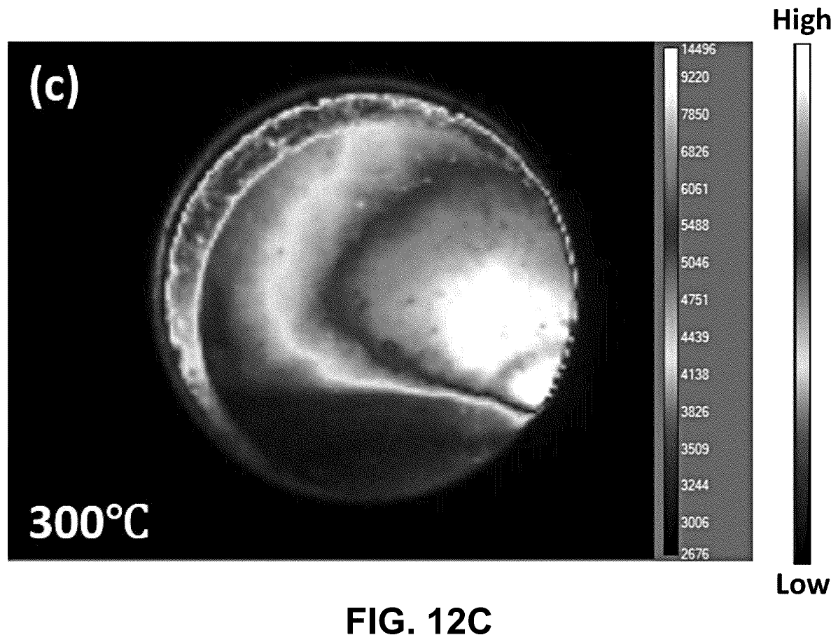

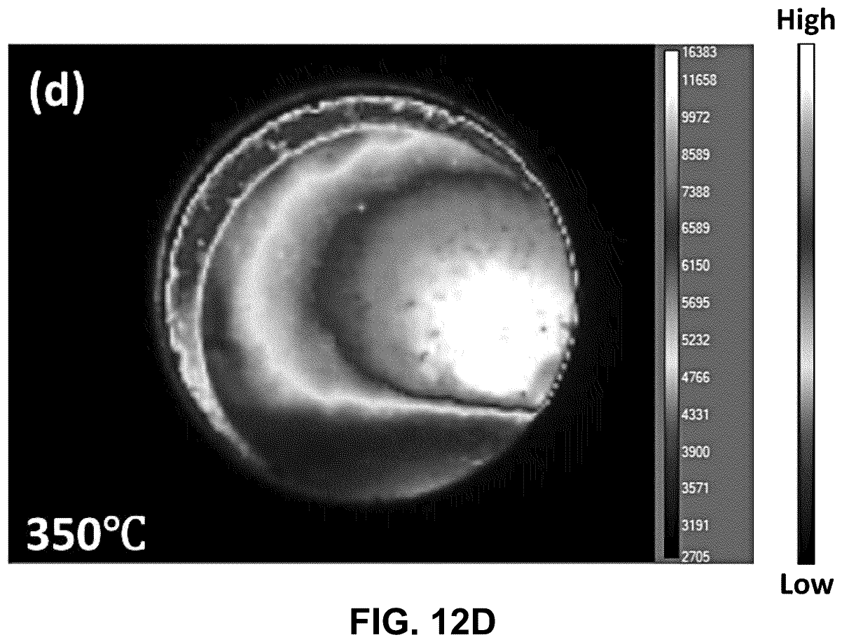

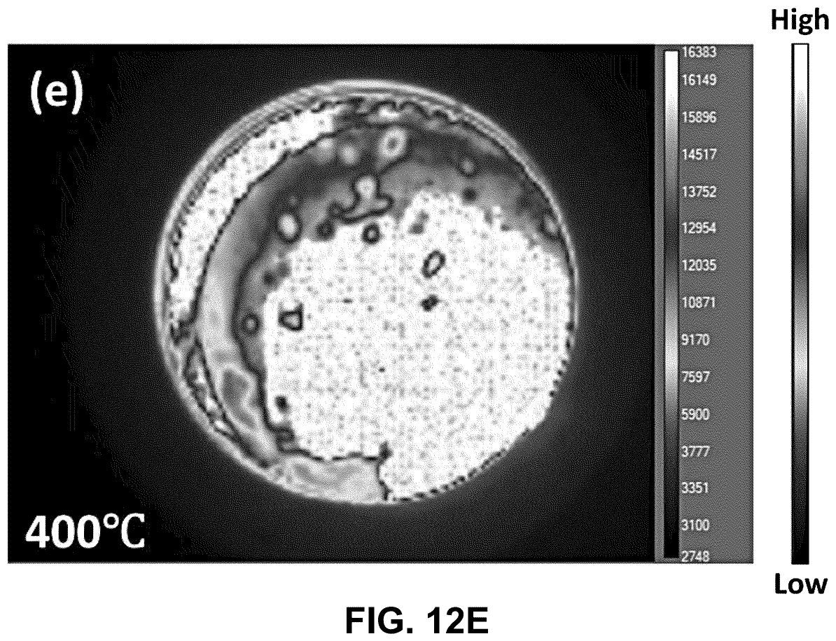

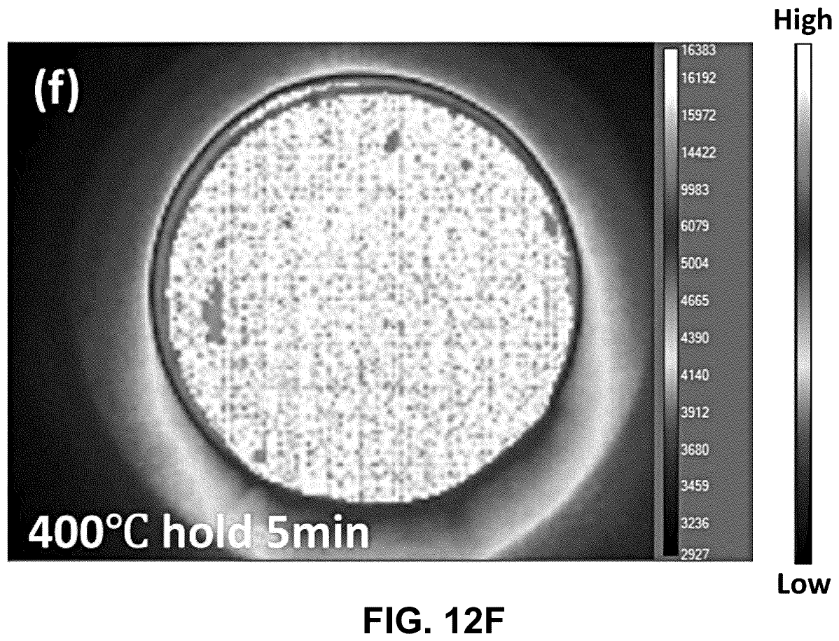

The reaction temperature, ramping rate, and time-on-stream were automatically controlled by the CBA Eurotherm controller, which adjusted the power supplied to the sample. A crystal detector measured the reflected power. The heating process, forward, and reflected power were recorded throughout the upcycling process. As shown in FIG. 11, during the initial dwell segment (approximately 1 minute), autotuning regulated the temperature at 160° C. The ramping rate of 20° C./min was maintained by adjusting the forward power, which fluctuated as the reaction progressed and the microwave sensitivity of the reactants changed. Initially, the microwave power was high as the temperature began to rise; once the target temperature was reached, the power rapidly decreased and stabilized during the hold segment. The hold time at the target temperature was 30 minutes. The external surface temperature of the sample was measured using an infrared thermometer, while a FLIR infrared camera captured thermal images to monitor temperature distribution and changes throughout the reaction, as shown in FIG. 1A and FIGS. 12A-12F.

The PP waste was first crushed and sieved to a particle size of less than 2 mm, then mixed with the catalyst in a 1:1 mass ratio before being loaded into the quartz tube reactor. The plastic/catalyst mixture was purged with nitrogen (N₂) at a flow rate of 50 mL/min for 30 minutes prior to initiating microwave irradiation. The resulting gases were collected in a gas bag, with an additional N₂ flow rate of 20 mL/min used as the carrier gas. Gas products were analyzed using a 4-channel Inficon Fusion micro gas chromatograph (Micro-GC) (FIG. 1A), and a cold trap was utilized to capture the liquid products.

Gas-phase products were analyzed using a four-channel Inficon Fusion micro-gas chromatograph (Micro-GC) (FIG. 1A), and a cold trap was utilized to capture the liquid products. The system was equipped with an Rt-Molsieve 5A column for O₂, N₂, H₂, CO, and CH₄; an Rt-U-Bond column for CO₂, C₂H₆, C₂H₄, and C₂H₂; an Alumina-Na₂SO₄ column for C₃H₈, C₃H₆, C₄H₁₀, and C₄H₈; and an Rxi-1 ms column for aromatic hydrocarbons including benzene and toluene. Notably, BTX products are theoretically in liquid phase under ambient temperature, but due to the existence of N₂ carrier gas flow, there still are some BTX products such as benzene and toluene that have been purged through the pipe towards sampling gas bag and Micro-GC. Hence, during the reaction we use heating tape to keep the temperature of the pipeline at around 120° C. (as shown in FIG. 1A), which means that the rest of BTX products such as benzene, toluene can go through the pipeline then enter Micro-GC because the boiling point of benzene and toluene are 80.1° C. and 111° C., respectively. Gas composition was recorded at 3-minute intervals throughout the reaction.

The mass of gases produced, including H₂ and C₁-C₈ hydrocarbons, was calculated by measuring the mass difference between the initial mixture (catalyst and plastic) and the post-reaction sample. Gas yield was determined as the ratio of the gas mass to the total mass of plastic feedstock, as shown in Eq. (1). The proportion of residue (comprising solid, wax, coke, and unreacted plastic) was calculated from the mass difference between the total solid residue (after extraction) and the catalyst, relative to the initial plastic mass, as detailed in Eq. (2).

Liquid products were collected by dissolving the residue in CHCl₃ and qualitatively analyzed using PerkinElmer gas chromatography-mass spectrometry (GC-MS). Due to the volatility of the liquid products and their tendency to adsorb onto the residue or adhere to the reactor walls, the liquid yield was estimated as 100% minus the gas yield and residue percentage, as shown in Eq. (3).

The yield of each product or phase (gas, liquid, or residue) was calculated as the ratio of the product mass to the total plastic mass, as detailed in Eq. (4). Conversion efficiency was determined using Eq. (5). Gas-phase product selectivity was expressed as the ratio of the mass of each gas product to the total mass of all gas-phase products, as outlined in Eq. (6).

For comparison, the catalytic upcycling of PP waste was also conducted under conventional thermal heating conditions, following the same procedure as in the DMCU system.

$$Gas \% ( wt \% ) = \frac{{Mass}_{( Catalyst + Plastic )} - {Mass}_{Afterreaction ( Catalyst + Plastic )}}{{Mass}_{Plastic}} \times 100 \% \qquad Eq . ( 1 )$$

$$Residue \% ( wt \% ) = \frac{{Mass}_{Afterextraction ( Catalyst + Plastic )} - {Mass}_{catalyst}}{{Mass}_{Plastic}} \times 1 \; 00 \% \qquad Eq . ( 2 )$$

$$Liquid \% ( wt \% ) = 100 \% - Gas \% - Residue \% \qquad Eq . ( 3 )$$

$$Yield \% ( wt \% ) = \frac{{Mass}_{product}}{{Mass}_{Plastic}} \times 1 \; 00 \% \qquad Eq . ( 4 )$$

$$Conversion \% = 100 \% - \frac{{Mass}_{unreacted plastic}}{{Mass}_{Plastic}} \times 100 \% \qquad Eq . ( 5 )$$

$$Selectivity \% = \frac{{Mass}_{product in gas phase}}{\sum {Mass}_{gas product}} \times 100 \% \qquad Eq . ( 6 )$$

For comparison, the catalytic upcycling of PP waste was also conducted under conventional thermal heating conditions, following the same procedure as in the DMCU system.

Characterization. A FLIR A6261 infrared thermal imaging camera was used to monitor the temperature distribution across the microwave catalyst bed. Positioned 0.5 meters from the quartz waveguide port, the camera captured thermal images and videos data, which was then analyzed using FLIR Research Max software to provide pixel-level temperature measurements.

X-ray diffraction (XRD) analysis was carried out using a PANalytical instrument with a CuKα radiation source, operating at 40 kV and 40 mA. The diffraction patterns were recorded over a 2θ range of 10° to 90° at a scanning speed of 5° per minute. Raman spectra of the samples were collected by Renishaw InVia Raman Microscope with <1-μm lateral spatial resolution. X-ray photoelectron spectroscopy (XPS) was performed using a PHI 5000 Versaprobe Scanning ESCA Microprobe spectrometer, equipped with a monochromatic Al Kα X-ray source (100 μm, 12.5 W, 15 kV). Sample surfaces were sputtered with an Ar ion gun (2 kV, 2 μA, 1×1 raster) at a sputtering rate of 17 nm/min. Binding energies were calibrated against the C 1s peak of adventitious carbon, set at 284.8 eV.

The acidity of the catalysts was evaluated through ammonia TPD (NH₃-TPD) using a Micromeritics Autochem HP 2950 instrument. The catalyst was initially heated to 300° C. at a rate of 10° C./min and held at this temperature for 30 minutes, followed by cooling to 50° C. During the cooling phase, a 15% NH₃ in He mixture was introduced at 10 mL/min for 30 minutes. The system was then purged with helium at the same flow rate for 30 minutes to ensure an inert environment. Finally, the sample was heated from 50° C. to 800° C. at 5° C./min to observe NH₃ desorption.

In-situ FTIR spectroscopy was employed to monitor product evolution during catalytic pyrolysis. The transmission cell was positioned between the IR source and a DTGS KBr detector. Pellets, composed of the PP plastic feedstock, Ru/Fe₂O₃ catalyst, and KBr, were compressed into thin discs and placed at the cell's center. The chamber was purged with N₂ (5 mL/min) for 1 hour to ensure an inert atmosphere. The temperature was ramped to 500° C. at 20° C./min, followed by a 30-minute dwell without gas flow. FTIR spectra were periodically collected to track functional group changes in both the pellet and the surrounding environment.

In-situ Raman spectroscopy was used to monitor product evolution during catalytic pyrolysis. The sample, including the PP plastic and Ru/Fe₂O₃ catalyst, was placed in a reaction cell with transparent windows to allow laser penetration. The reaction cell was purged with N₂ gas (5 mL/min) for 1 hour to create an inert atmosphere. The temperature was gradually raised to 500° C. at a rate of 20° C./min, with Raman spectra collected at regular intervals. These spectra captured changes in functional groups within the sample, providing real-time insights into bond breakage and formation during the pyrolysis process.

The internal morphologies of the prepared samples were examined using a JEOL JEM-2100 high-resolution TEM, operated at 200 kV. TGA was utilized to analyze the PP samples and solid residues. The heating process was carried out from 50° C. to 900° C. at a controlled rate of 10° C./min, under a nitrogen atmosphere with a flow rate of 100 mL/min.

DFT Methodology. The computational investigations were carried out using spin-polarized DFT with the Vienna Ab initio Simulation Package (VASP, version 6.2.3) (Ref. 47). For electron-ion interactions, the projector-augmented wave (PAW) method was utilized alongside the generalized gradient approximation (GGA) employing the Perdew-Burke-Ernzerhof (PBE) functionals to calculate electron exchange and correlation energies (Refs. 48 and 49). The Grimme's DFT-D3 dispersion correction was used to better describe the weak interactions within the system (Ref. 50). Plane wave basis sets were defined with a cutoff energy of 400 eV. A Monkhorst-Pack k-point grid of 1×1×1 was utilized. A slab was derived from the optimized bulk structure of α-Fe₂O₃ along the [110] orientation, employing a p(2×2) supercell with four layers. A 15 Å thick vacuum layer was included to prevent periodic electrostatic interaction. During surface optimization, the top two layers and adsorbates were entirely relaxed, while the bottom two layers remained fixed, until forces converged below 0.03 eV/Å. Transition state searches used the climbing image nudged elastic band (CINEB) or dimer techniques (Ref. 51), with an atomic force convergence criterion of 0.05 eV/Å, and were validated to have a single imaginary frequency through vibrational frequency analyses.

Characterization Results. As shown in FIGS. 12A-12F, the thermal images reveal the dynamic temperature distribution during microwave heating, showcasing the selective and progressive heating behavior of the catalyst-PP particle mixture. Initially, at 200° C., localized hot spots form within the cooler blue regions, indicating that the microwave selectively heats the microwave-sensitive catalyst Ru/Fe₂O₃ while the PP, which does not absorb microwave energy, remains at a lower temperature. As the temperature rises to 250° C. and 300° C., these hot spots expand, gradually drawing the surrounding PP particles into the high-temperature zones, where they react more rapidly due to the proximity to the heated catalyst. By 350° C., the high-temperature regions dominate the mixture, indicating that most of the PP near the catalyst hot spots has been converted. At 400° C., nearly the entire mixture reaches a high temperature, with the remaining cooler regions diminishing, suggesting that the PP has been progressively and efficiently reacted. Finally, after holding at 400° C. for 5 minutes, the temperature distribution becomes uniform, confirming the complete reaction of all PP. This series of observations highlights the selective heating capability of microwave irradiation, enhancing the efficiency and selectivity of the catalytic upcycling process by creating localized high-temperature zones that facilitate the rapid conversion of PP.

The XRD patterns of sole α-Fe₂O₃ and fresh Ru/Fe₂O₃ (4 wt. % Ru) are shown in FIG. 9A. The characteristic reflections of hematite (α-Fe₂O₃) are present in both samples, as indicated by the JCPDS #02-0919 reference. This indicates that the α-Fe₂O₃ structure remains intact even after the deposition of Ru. However, slight changes in peak intensities in peak intensities in the Ru/α-Fe₂O₃ pattern suggest changes in crystallinity or minor structural modifications due to the incorporation of Ru. The absence of distinct Ru-related peaks is likely due to the high dispersion of Ru nanoparticles on the α-Fe₂O₃ surface and their small size, which makes them undetectable by XRD, a common challenge in materials with very fine or dispersed metal catalysts (Ref. 52).

The Raman spectra in FIG. 9B show the structural integrity of the catalyst before and after the reaction. Sole α-Fe₂O₃ exhibits characteristic Raman bands at 212, 276, 386, and 580 cm⁻¹ vibration peaks, while fresh Ru/Fe₂O₃ shows additional features, indicating slight structural modifications due to Ru deposition (Ref. 53). After the reaction, the spent Ru/Fe₂O₃ sample displays two notable peaks at 1294 cm⁻¹ and 1506 cm⁻¹, which are commonly attributed to carbonaceous deposits, such as graphite, that accumulate on the catalyst surface during the catalytic process.

The XPS spectra in FIG. 9C reveal the surface composition and chemical states of sole Fe₂O₃, fresh Ru/Fe₂O₃, and spent Ru/Fe₂O₃. For sole α-Fe₂O₃, peaks corresponding to Fe 2p and O 1s are evident, confirming the presence of iron oxide on the surface. In the fresh Ru/α-Fe₂O₃ catalyst, additional peaks corresponding to Ru 3d and C 1s are observed, indicating the successful deposition of Ru on the α-Fe₂O₃ surface. After the catalytic reaction, the XPS spectrum of the spent Ru/α-Fe₂O₃ shows a notable increase in the C 1s signal, which is consistent with the Raman findings from Figure S3 (b). This increase is likely due to the deposition of carbonaceous species during the reaction, which can lead to catalyst deactivation over time, as the accumulation of carbon on the catalyst surface can hinder its activity.

The NH₃-TPD profiles in FIG. 9D reveal differences in surface acidity between sole Fe₂O₃ and fresh Ru/Fe₂O₃, closely tied to their catalytic performance in PP upcycling. Sole Fe₂O₃ shows a single strong peak around 595° C., indicating the presence of strong acid sites but a lack of weak to moderate acid sites, limiting its effectiveness in the initial stages of plastic cracking (Ref. 54). In contrast, Ru/Fe₂O₃ exhibits two distinct peaks: one at 260° C., corresponding to weak/moderate acid sites introduced by Ru, and another at 765° C., reflecting enhanced strong acid sites. The presence of weak acid sites tends to facilitate β-scission reactions and plays a role in the initial adsorption and cracking of PP chains (Ref. 44), while stronger acid sites promote further cracking and dehydrogenation under the high temperatures associated with the DMCU system, leading to the efficient conversion of heavier hydrocarbons into valuable lighter products like propylene.

The in-situ FTIR spectra in FIG. 9E provide critical insights into the chemical evolution during the catalytic upcycling of PP over the Ru/Fe₂O₃ catalyst. Initially, at lower temperatures (25-300° C.), strong CH₂ and C—H stretching bands are observed, indicating the presence of intact polymer chains (Refs. 55 and 56). As the temperature rises to 400° C., these bands intensify, reflecting the breaking of PP chains and the formation of smaller hydrocarbon intermediates. At 500° C., the CH₂ and C—H peak intensities increase during the first 10 minutes, suggesting active chain scission. However, after extended holding at 500° C., these peaks gradually weaken and disappear, signifying the near-complete breakdown of PP into smaller hydrocarbons. Simultaneously, C═C and C—O stretching bands begin to emerge around 400° C. and grow stronger at higher temperatures, indicating the formation of olefins and oxygenated species through dehydrogenation and secondary reactions (Refs. 55 and 56). This sequential evolution—initial PP chain cracking followed by olefin formation—demonstrates the catalyst's role in promoting both the breakdown of polymer chains and the selective production of valuable olefins.

The in-situ Raman spectra in FIG. 9F provide complementary insights to the in-situ FTIR findings, revealing key transformations during the catalytic upcycling of PP over the Ru/Fe₂O₃ catalyst. Below 400° C., both CH₂ and CH₃ bands are prominent, indicating the intact polymer structure (Ref. 57). As the temperature approaches 400° C., these bands weaken and eventually disappear, reflecting the breakdown of PP chains—a trend also observed in the in-situ FTIR spectra. Simultaneously, the emergence of C═C stretching bands after 400° C. points to the formation of olefins, consistent with the C═C growth seen in the in-situ FTIR. This confirms that dehydrogenation reactions are actively producing valuable olefins, such as propylene and ethylene, during the reaction. Additionally, the development of C═O stretching bands further supports the formation of oxygenated species, likely due to surface reactions on the catalyst. Moreover, the increasing Fe—C bands in the in-situ Raman signify carbon deposition (Ref. 33). Together, these analyses provide a coherent understanding of the catalytic upcycling of PP: initial polymer chain scission is followed by selective olefin production, with both techniques highlighting the formation of C═C bonds and the eventual accumulation of carbon deposits. This combined insight underscores the efficiency of Ru/Fe₂O₃ in promoting polymer breakdown and light olefin formation.

TGA and DTG Analysis. The PP feedstocks and post-reaction residues were analyzed using TGA under an N₂ atmosphere. As shown in FIGS. 3A-3B, pure chemical PP, McDonald's PP, and mixed PP decompose completely before 500° C. The main decomposition of pure chemical PP occurs at 470° C., while real-world PP decomposes at a slightly lower temperature of 450° C.

Real-world, post-consumer PP decomposes at a slightly lower temperature, approximately 450° C. This shift suggests that additives or contaminants in the real-world PP may lead to earlier thermal degradation. The TGA and derivative thermogravimetric (DTG) results in FIGS. 3C-3D offer valuable information regarding the post-reaction residues from the DMCU process. The results show that the mass loss before 500° C. is minimal: 1.7% for mixed PP and 2.5% for both virgin chemical and McDonald's PP. This indicates that the DMCU reaction achieves near-complete conversion of the PP feedstocks, leaving only a small fraction of residue. These residues, which consist of solid or carbonaceous deposits, demonstrate the high efficiency of the DMCU system in breaking down PP into lighter hydrocarbon products while minimizing unconverted material. DTG analysis shows significant peaks at 214° C. and 437° C. for residues from McDonald's and virgin chemical PP, which indicates the breakdown of wax-like intermediates formed during the reaction (Ref. 16). These intermediates likely arise from partial thermal decomposition and dehydrogenation processes, resulting in heavier hydrocarbons that degrade at these specific temperatures. Beyond 500° C., the observed mass loss is attributed to the reduction reaction between the carbon deposits and the Ru/α-Fe₂O₃ catalyst. This reduction typically occurs at high temperatures under a nitrogen atmosphere, as evidenced by mass loss peaks at approximately 834° C. for mixed and virgin PP residues and 840° C. for McDonald's PP residues. The slightly higher mass loss observed in real-world PP residues compared to pure PP suggests greater carbon accumulation on the catalyst during the upcycling of real-world PP, which necessitates a marginally higher temperature for complete reduction. This underscores the influence of feedstock composition on carbon deposition behavior and catalyst interaction.

TEM and SEM-EDX Mapping. FIGS. 4A-4F shows TEM and HRTEM analyses of the fresh and spent 4 wt. % Ru/α-Fe₂O₃ catalysts. In the fresh state, well-defined agglomerates (˜200 nm) with clean surfaces are observed (FIGS. 4A-4B). HRTEM (FIG. 4C) reveals lattice spacings of 0.318 nm (RuO2 (110)) and 0.251/0.270 nm (Fe₂O₃ (110)/(104)), confirming well-dispersed RuO₂ on the support.

After reaction, the spent catalyst retains its morphology (FIG. 4D) but shows blurred particle boundaries. High-magnification TEM (FIG. 4E) highlights localized carbon deposits, consistent with SEM-EDX mapping (FIG. 16F) showing uniformly distributed carbon. HRTEM (FIG. 4F) indicates RuO₂ is reduced to metallic Ru (0.135 nm, (110)), accompanied by graphitic carbon (0.192 nm, (101)) and α-Fe₂O₃ (0.145 nm (300), 0.221 nm (113)). The reduction from larger RuO2 crystallites to smaller Ru nanoparticles occurs without obvious migration or aggregation, aligning with XPS results

Raman Analysis. The Raman spectra in FIG. 5 provide detailed insights into the structural integrity and surface changes of the Ru/Fe₂O₃ catalyst before and after microwave-assisted polypropylene upcycling, specifically comparing reactions with pure PP and real-life PP. In the fresh Ru/Fe₂O₃ catalyst, prominent peaks below 600 cm⁻¹ represent the characteristic lattice vibrations of Fe₂O₃, indicating the intact iron oxide structure (Ref. 53). After the reaction, the spent Ru/Fe₂O₃ catalyst used in the pure PP reaction still retains these Fe₂O₃ peaks. Weak carbon-related bands at 1294 cm⁻¹ and 1506 cm⁻¹ also appear, indicating limited carbon deposition. In contrast, the spent Ru/Fe₂O₃ catalyst from the real-life PP reaction shows more pronounced D and G bands at 1324 cm⁻¹ and 1577 cm⁻¹, corresponding to disordered and graphitic carbon, respectively, signifying a higher level of carbon accumulation on the catalyst surface. This increase in carbon deposition correlates with the decreased intensity of the Fe₂O₃ peaks, suggesting that the surface may be partially obscured or modified by the deposited carbon, likely due to impurities and additives in real-life PP.

XPS Analysis. XPS analysis of the Fe 2p, Ru 3d, and O 1s regions reveals significant changes in the oxidation states and surface composition of the Ru/Fe₂O₃ catalyst before and after microwave-assisted PP upcycling. As shown in FIG. 6A, the Fe 2p spectra of the fresh catalyst display both Fe²⁺ and Fe³⁺ species, with a notable presence of Fe²⁺, indicating partial reduction. After the reaction, the Fe²⁺ signal diminishes, while the Fe³⁺ signal intensifies, suggesting a shift toward higher oxidation states.

As illustrated in FIG. 6B, the Ru 3d spectra show Ru 3d_{5/2} and Ru 3d_{3/2} peaks due to spin-orbit splitting. The Ru 3d_{3/2} peak overlaps with the C 1s peak at ˜284.8 eV, attributed to CHₓ impurities on the sample surface. Therefore, only the Ru 3d_{5/2} binding energies are discussed (Refs. 58 and 59). The Ru 3d spectra of the fresh catalyst indicates a mixture of Ru⁰ and Ru⁴⁺, while the spent catalyst shows an increased Ru⁰ component and a decreased Ru⁴⁺, reflecting the oxidation state changes of ruthenium during the reaction.

As shown in FIG. 6C, the O 1s spectra of the fresh catalyst show contributions from lattice oxygen (Oₐ), surface oxygen vacancies (Oᵥ), and adsorbed oxygen species (O_{c}). In contrast, the spent catalyst displays an increase in Oᵥ and a decrease in Oₐ, indicating shifts in surface oxygen species. These results highlight the oxidation state changes in Fe and Ru, as well as the evolving nature of oxygen species on the catalyst surface after the upcycling process.

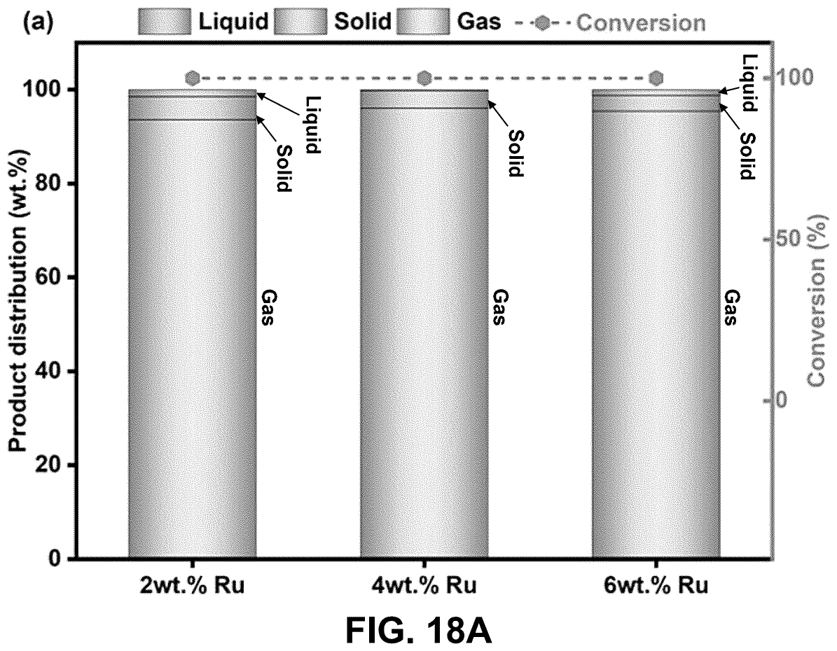

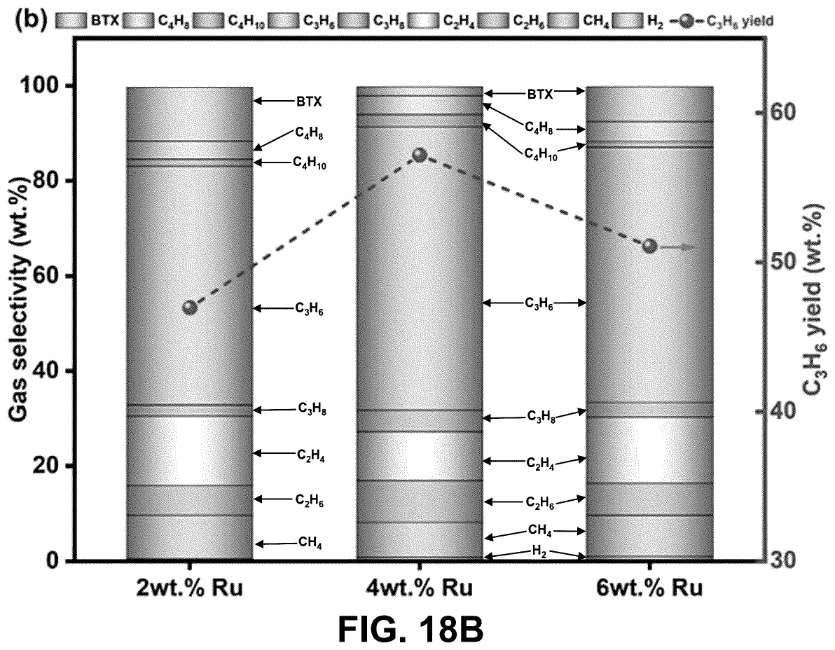

Optimization of Ru Loading. To determine an optimal Ru loading considering both catalytic performance and noble-metal usage, 2, 4, and 6 wt. % Ru/α-Fe₂O₃ catalysts were compared for microwave-assisted PP upcycling at 400° C. As shown in FIG. 18A, all catalysts achieved nearly complete PP conversion with gas fractions above 93 wt. %, indicating that even low Ru loadings can effectively catalyze PP cracking under microwave irradiation. However, as shown in FIG. 18B, gas-phase product selectivity varied markedly: 4 wt. % Ru delivered the highest C₃H₆ selectivity (59.6 wt. %) and yield (57.2 wt. %), outperforming both 2 wt. % Ru (50.2 wt. % selectivity, 46.9 wt. % yield) and 6 wt. % Ru (53.5 wt. % selectivity, 51.1 wt. % yield). The different performance of 4 wt. % Ru is attributed to an optimal balance between the density of Ru—Fe interfacial sites and the suppression of non-selective hydrogenolysis or over-cracking, which are more pronounced at both lower and higher Ru loadings. Since the 4 wt. % Ru/α-Fe₂O₃ catalyst showed the highest absolute C₃H₆ yield and selectivity among the tested loadings, it was chosen as the representative catalyst in this Example.

Conclusion. This Example demonstrates the effectiveness of a microwave-assisted catalytic upcycling process for PP waste, utilizing a microwave-sensitive Ru/α-Fe₂O₃ catalyst and emphasizing the critical synergy between microwave energy and the microwave-responsive catalyst. In comparison to conventional thermal methods, this system achieves enhanced propylene selectivity and higher conversion efficiency at lower temperatures, with significantly reduced by-product formation. Mechanistic analysis reveals that Fe—O sites are more proficient in C—H bond activation, facilitating dehydrogenation, while Ru—O sites promote selective C—C bond cleavage and hydrogenation, which aid in the formation of valuable propylene. The interaction between microwave energy and Ru/α-Fe₂O₃ enhances reaction control, accelerating C—C bond dissociation and effectively suppressing undesired secondary cracking. Despite minor deactivation due to carbon deposition, the system demonstrates excellent stability over multiple reaction cycles, indicating strong potential for scalability.

### 2. Continuous Feeding Microwave Reactor System

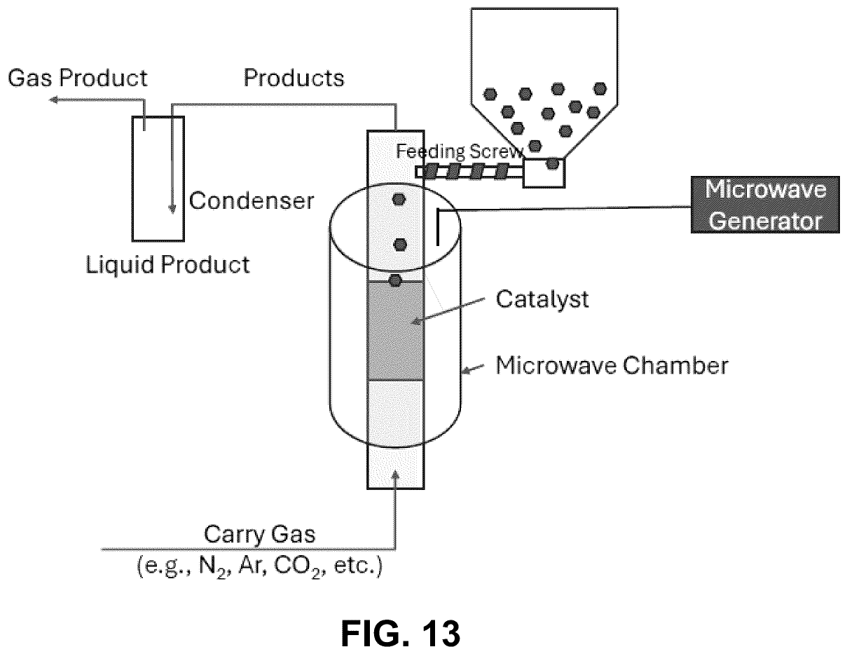

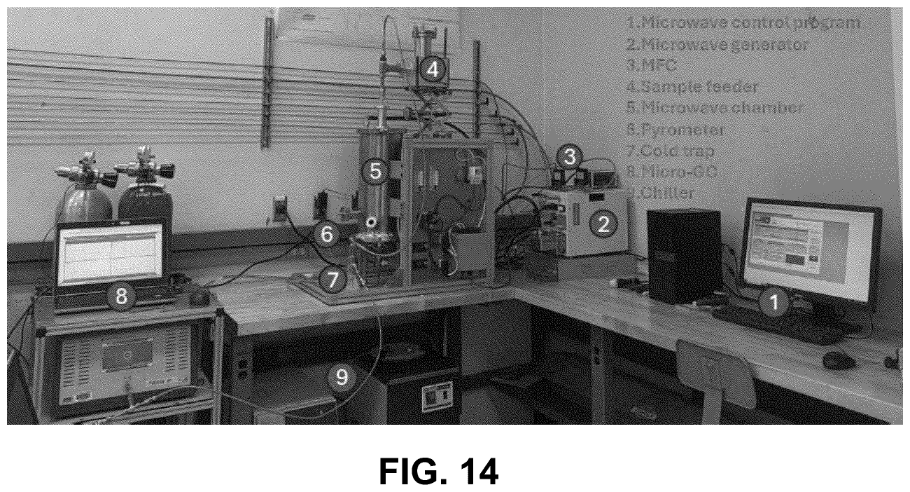

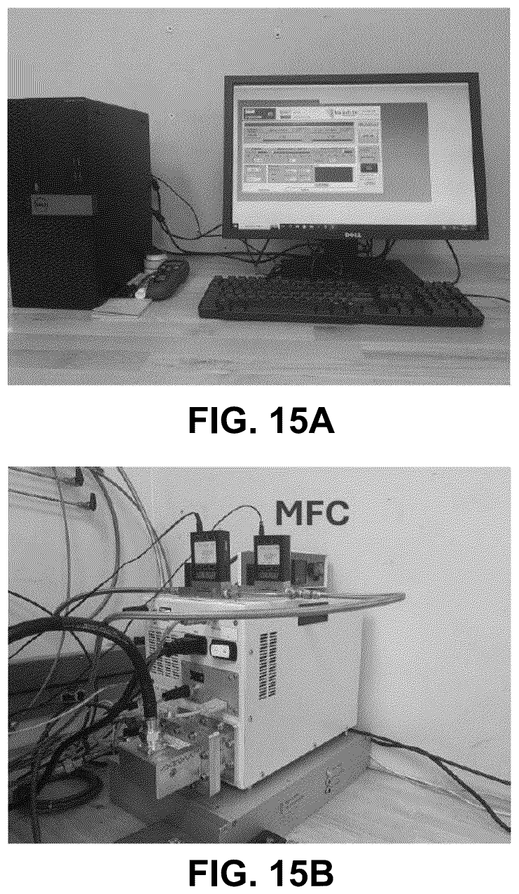

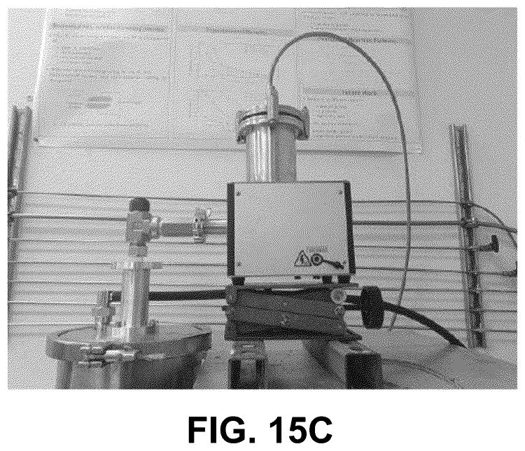

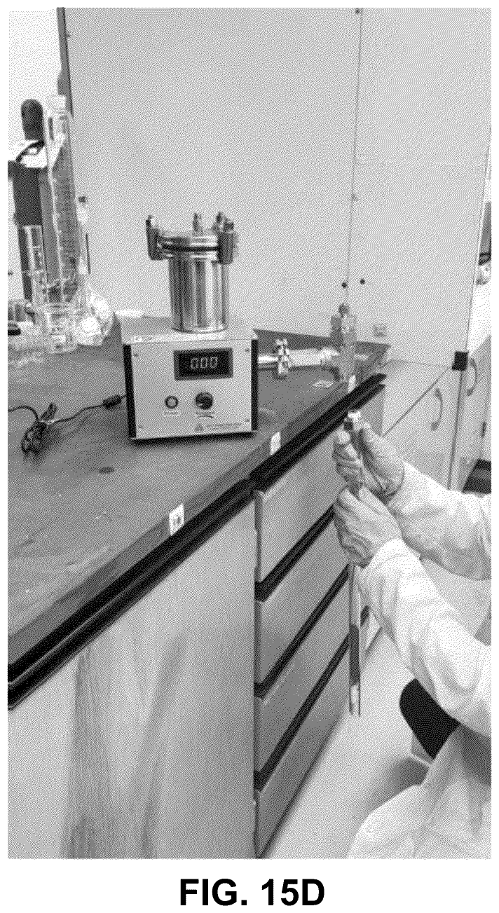

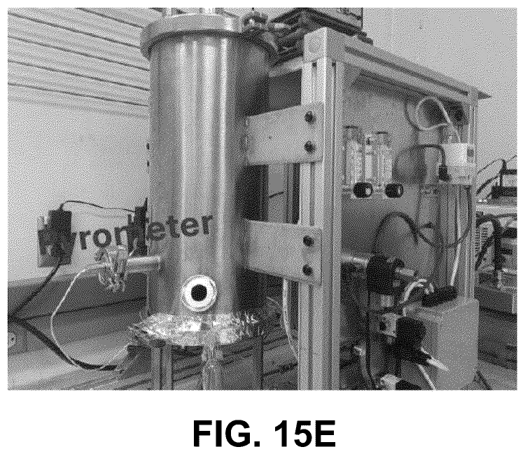

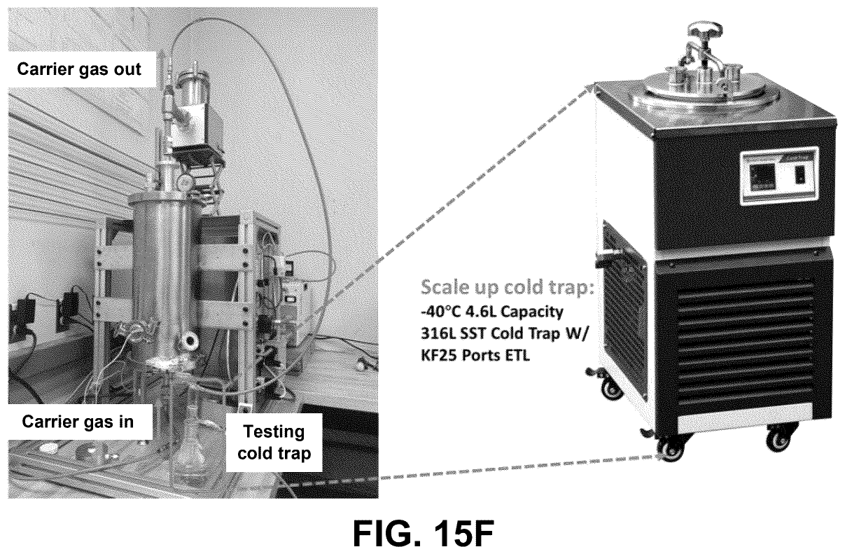

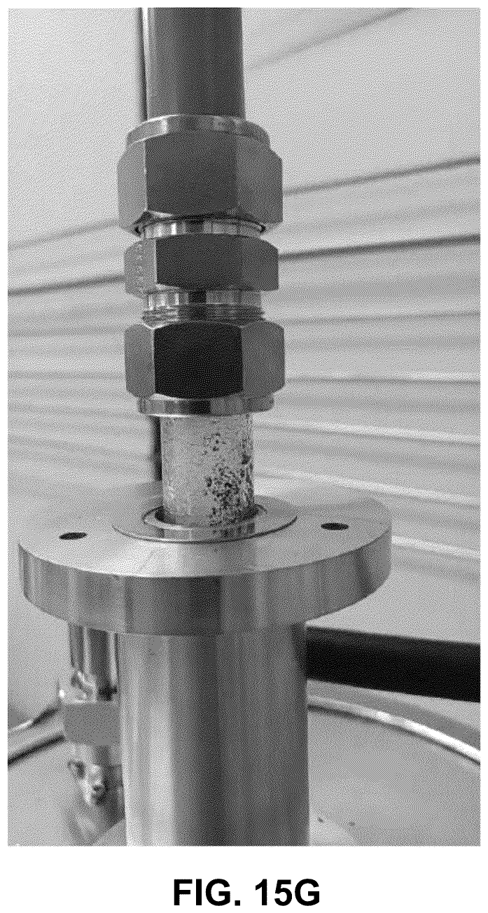

FIG. 13, FIG. 14, and FIGS. 15A-15G depict a continuous feed microwave reactor system, including a continuous feeding system and a microwave catalytic reactor. The system, in combination with a stable microwave-sensitive catalyst, can be used for production of valuable products (e.g., light olefins, aromatics, hydrogen, carbon nanotubes and/or nanofibers, and the like). The system is tunable to adjust for selectivity to desired products. The system can be operated at a relatively low reaction temperature (e.g., less than 500° C., less than 400° C., or less than 300° C.).

This system utilizes microwave technology to achieve rapid and selective heating, thereby reducing energy consumption and improving reaction efficiency. The continuous design allows for the scalable processing of large volumes of waste, making it suitable for industrial applications. Microwave-assisted catalysis offers distinct advantages over conventional thermal methods, including precise control over reaction conditions, which can lead to higher selectivity and yield of desired products. This system overcomes the limitations of traditional pyrolysis by lowering the reaction temperatures required, thereby reducing the carbon footprint and minimizing the production of unwanted by-products. The design allows for plastic solids to be continuously fed into the microwave reactor, and with a high stable catalyst, scaleup plastic upcycling under microwave conditions can be achieved.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A method, comprising:
contacting an M1/support catalyst with a plastic feedstock in a reaction zone;
purging the reaction zone with an inert gas; and
heating the M1/support catalyst by microwave irradiation to a target temperature, thereby producing a gaseous product;
wherein the support catalyst is selected from zeolite, CeO₂, ZrO₂, and α-Fe₂O₃;
wherein M1 is a metal nanoparticle selected from Zr, Ga, Cs, Pt, Pd, Ru, and a combination thereof.

2. The method of claim 1, wherein the M1/support catalyst is a fixed-bed catalyst.

3. The method of claim 1, wherein contacting the M1/support catalyst with the plastic feedstock comprises mixing the plastic feedstock with the M1/support catalyst in a mass ratio of about 0.25:1 to about 3:1 (plastic feedstock: M1/support catalyst).

4. The method of claim 1, wherein the plastic feedstock comprises a solid plastic.

5. The method of claim 4, wherein the plastic feedstock comprises particles of plastic having a particle size of about 0.25 mm to about 10 mm.

6. The method of claim 1, wherein the plastic feedstock comprises a melted plastic.

7. The method of claim 1, wherein the plastic feedstock comprises a polyolefin plastic.

8. The method of claim 7, wherein the gaseous product comprises a light olefin gas.

9. The method of claim 8, wherein the light olefin gas comprises from about 40 wt % to about 100 wt % of the gaseous product.

10. The method of claim 8, wherein the light olefin gas has a product yield of about 40% to about 100%, wherein the product yield is calculated as the ratio of the mass of light olefin gas produced to the mass of the plastic feedstock.

11. The method of claim 8, wherein the light olefin gas comprises a propylene gas.

12. The method of claim 1, wherein the catalyst comprises the metal nanoparticle disposed on the support catalyst at a weight percentage of about 1% to about 6%.

13. The method of claim 1, wherein the support catalyst is selected from Y-type zeolite, a pentasil aluminosilicate zeolite, a mordenite zeolite, a beta zeolite, CeO₂, ZrO₂, and α-Fe₂O₃.

14. The method of claim 1, wherein the target temperature is about 300° C. to about 500° C.

15. The method of claim 1, wherein the M1/support catalyst is heated at a rate of about 10° C./min to about 30° C./min until reaching the target temperature.

16. The method of claim 1, wherein the M1/support catalyst is held at the target temperature for about 0.5 minutes to about 1 hour.

17. A system, comprising:
a microwave chamber, comprising a catalyst, a microwave inlet, a sample inlet, a gas inlet, and an outlet;
a microwave generator, configured to generate microwave radiation and deliver the microwave radiation to the microwave chamber via the microwave inlet;
a sample feeder, comprising a plastic feedstock and configured to deliver the plastic feedstock from to the microwave chamber via the sample inlet;
a gas delivery device, comprising an inert gas and in gaseous communication with the microwave generator via the gas inlet; and
a condenser, in gaseous communication with the microwave chamber via the outlet and configured to collect a gaseous product produced in the microwave chamber;
wherein the catalyst is an M1/support catalyst;
wherein the support catalyst is selected from zeolite, CeO₂, ZrO₂, and α-Fe₂O₃; and
wherein M1 is a metal nanoparticle selected from Zr, Ga, Cs, Pt, Pd, Ru, and a combination thereof.

18. The system of claim 17, wherein the support catalyst is selected from Y-type zeolite, a pentasil aluminosilicate zeolite, a mordenite zeolite, a beta zeolite, CeO₂, ZrO₂, and α-Fe₂O₃.

19. The system of claim 17, wherein the catalyst comprises the metal nanoparticle disposed on the support catalyst at a weight percentage of about 1% to about 6%.

20. The system of claim 17, further comprising a pyrometer;
wherein the plastic feedstock delivered to the microwave chamber forms a reaction mixture with the catalyst; and
wherein the pyrometer is configured to measure the temperature of the reaction mixture.
